(19) United States
(12) Patent Application Publication
(10) Pub. No.: US 2007/0155791 A1
(43) Pub. Date: Jul. 5, 2007

(54) Methods for treating cutaneous lupus using aminoisoindoline compounds

(76) Inventors: Jerome B. Zeldis, Princeton, NJ (US); Patricia E.W. Rohane, Florham Park, NJ (US); Peter H. Schafer, Somerset, NJ (US)

Correspondence Address: JONES DAY, 222 EAST 41ST ST, NEW YORK, NY, 10017, US

(21) Appl. No.: 11/645,319

(22) Filed: Dec. 21, 2006

Related U.S. Application Data
(60) Provisional application No. 60/754,795, filed on Dec. 29, 2005.
(60) Provisional application No. 60/755,246, filed on Dec. 29, 2005.
(60) Provisional application No. 60/787,436, filed on Mar. 30, 2006.

(51) Int. Cl. A61K 31/454; A61K 31/4035

(52) U.S. Cl. 514/323; 514/417

(57) ABSTRACT

Methods of treating cutaneous lupus in a human are disclosed. Specific methods encompass the administration of (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione (ACTIMID™), 3-(4-amino-1-oxo-1,3-dihydro-isoindol-2-yl)-piperidine-2,6-dione (REVLIMID®), or cyclopropyl 2-[(1S)-1-(3-ethoxy-4-methoxyphenyl)-2-(methylsulfonyl)ethyl]-3-oxoisoindolin-4-yl}carboxamide, alone or alternatively, in combination with a second active agent.

This application claims the benefit of U.S. provisional application Nos. 60/754,795, filed Dec. 29, 2005, 60/755,246, filed Dec. 29, 2005, and 60/787,436, filed Mar. 30, 2006, the contents of which are incorporated by reference herein in their entirety.

### 1. FIELD OF THE INVENTION

This invention provides methods of treating, preventing and/or managing cutaneous lupus by the administration of (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione (ACTIMID™), 3-(4-amino-1-oxo-1,3-dihydro-isoindol-2-yl)-piperidine-2,6-dione (REVLIMID®), or cyclopropyl 2-[(1S)-1-(3-ethoxy-4-methoxyphenyl)-2-(methylsulfonyl)ethyl]-3-oxoisoindolin-4-yl}carboxamide, alone or in an alternative embodiment in combination with other therapeutics.

The invention also provides pharmaceutical compositions and dosage forms comprising (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl {2-[(1S)-1-(3-ethoxy-4-methoxyphenyl)-2-(methylsulfonyl)ethyl]-3-oxoisoindolin-4-yl}carboxamide, alone or in combination with other therapeutics for use in methods of treating, preventing and/or managing cutaneous lupus.

### 2. BACKGROUND OF THE INVENTION

Lupus or lupus erythematosus is an autoimmune disorder that can cause chronic inflammation in various parts of the body, especially the skin, joints, blood, and kidneys. The body's immune system normally makes proteins called antibodies to protect the body against viruses, bacteria, and other foreign materials (i.e., antigens). In an autoimmune disorder such as lupus, the immune system loses its ability to tell the difference between antigens and its own cells and tissues and can make antibodies directed against its own cells and tissues to form immune complexes. These immune complexes can build up in the tissues and cause inflammation, injury to tissues and/or pain. The three most common types of lupus include systemic lupus erythematosus (SLE), cutaneous lupus erythematosus (CLE) and drug-induced lupus. More detailed descriptions of lupus or lupus erythematosus can be found in Wallace, 2000, The Lupus Book: A Guide for Patients and Their Families, Oxford University Press, Revised and Expanded Edition, which is incorporated by reference herein in its entirety.

Systemic lupus erythematosus (SLE) is an autoimmune disease involving multiple organ systems that is defined clinically and associated with antibodies directed against cell nuclei. SLE can affect any system or organ in the body including the joints, skin, lungs, heart, blood, kidney, or nervous system. Symptoms of SLE can range from being a minor inconvenience to very serious and even life threatening. For example, a SLE patient may experience (a) no pain or extreme pain, especially in the joints; (b) no skin manifestations or disfiguring rashes; and/or (c) no organ involvement or extreme organ damage. As discussed above, many clinical manifestations of SLE are caused by the effects of immune complexes on various tissues or cell surface components. However, it is still unclear whether polyclonal B-cell activation or a response to specific antigens exists. Nonetheless, a genetic predisposition to the development of SLE may exist. More detailed descriptions of SLE can be found in Lahita, 1999, Systemic Lupus Erythematosus, Academic Press, Third Edition, which is incorporated by reference herein in its entirety.

Drug-induced lupus generally occurs after the use of certain prescribed drugs. The symptoms of drug-induced lupus are similar to those of SLE. The drugs most commonly connected with drug-induced lupus are hydralazine (used to treat high blood pressure or hypertension) and procainamide (used to treat irregular heart rhythms). However, only an extremely small number who take these drugs can develop overt drug-induced lupus. The symptoms usually fade when the medications are discontinued.

Cutaneous lupus or cutaneous lupus erythematosus affects primarily the skin and is generally characterized by skin inflammation, skin rashes and hemorrhages in the skin. Cutaneous lupus may also affect hair and mucous membranes but usually does not involve internal organs like SLE. Cutaneous lupus can be categorized into groups including acute cutaneous lupus erythematosus (ACLE), subacute cutaneous lupus erythematosus (SCLE), chronic cutaneous lupus erythematosus (CCLE) or discoid lupus erythematosus (DLE) and neonatal lupus erythematosus (NLE). More detailed descriptions of cutaneous lupus or cutaneous lupus erythematosus can be found in Kuhn et al., 2004, Cutaneous Lupus Erythematosus, Springer, First Edition, which is incorporated by reference herein in its entirety.

ACLE is generally a photosensitive dermatosis. It can appear as flattened areas of red skin that resemble a persistent sunburn or have a rash-like appearance. ACLE may erupt in a butterfly pattern localized to the central portion of the face and/or in a generalized pattern including other areas such as the arms, legs and body. The etiology of ACLE is believed to be multi-factorial, involving genetic, environmental and hormonal factors. In patients who are predisposed genetically, ACLE can be triggered by viruses (e.g., EBV) and exposure to ultraviolet light.

SCLE is a non-scarring non-atrophy-producing photosensitive dermatosis. In some cases, SCLE appears as a non-itchy ring-shaped dry rash on the upper back and chest, often following sun exposure. SCLE may occur in patients with systemic lupus erythematosus, Sjögren syndrome and deficiency of the second component of complement (C2d) or it can be drug induced. SCLE usually occurs in genetically predisposed individuals, most often in patients with human leukocyte antigen B8 (HLA-B8), human leukocyte antigen DR3 (HLA-DR3), human leukocyte antigen DRw52 (HLA-DRw52) and human leukocyte antigen DQ1 (HLA-DQ1). SCLE strongly associates with anti-Ro (SS-A) autoantibodies. Usually, SCLE manifests following UV light exposure, but other triggers or inciting factors are also implicated.

CCLE or DLE is a chronic, scarring, atrophy producing, photosensitive dermatosis. DLE commonly appears as red scaly patches which leave white scars. DLE predominantly affects the cheeks and nose, but sometimes involves the upper back, neck, backs of hands, bald areas in scalp and the lips. DLE may occur in patients with systemic lupus erythematosus (SLE). Some patients also have the lesions of SCLE and some may have a malar rash. Therapy with sunscreens, topical corticosteroids and antimalarials can be effective. DLE probably occurs in genetically predisposed individuals, but the exact genetic connection has not been determined. The pathophysiology of DLE is not well understood. It has been suggested that a heat shock protein is induced in the keratinocyte following ultraviolet (UV) light exposure or stress and this protein may act as a target for γδ T-cell-mediated epidermal cell cytotoxicity.

Verrucous DLE, lupus profundus, mucosal DLE, palmar-plantar DLE and lupus tumidus are some specific forms of DLE. Verrucous DLE refers to DLE having lesions that can develop into very thick scales. Lupus profundus refers to DLE having lesions that may occur in conjunction with firm lumps in the fatty tissue underlying the skin. Mucosal DLE refers to the lesions that occasionally occur in the mucus membranes of the mouth, nose and eyes. Palmar-plantar DLE refers to the lesions that occasionally occur on the hands and feet. Lupus tumidus appears as smooth, shiny, red-violet plaques of the head and neck that can be pruritic and have a fine scale. The lupus tumidus lesions usually clear without scarring and can recur in their original distribution.

NLE is a rare condition in children and usually appears as nonscarring, non-atrophy-producing lesions. In some cases, newborn babies born to mothers with SCLE may develop NLE with a temporary ring-like or annular rash. NLE is believed to be related to various factors including genetic predisposition, viral infection and other unknown factors. NLE may affect the skin, heart, liver, blood-forming elements or the spleen.

Lupus erythematosus (LE) of childhood relates to genetic factors and perhaps other environmental events. LE of childhood may affect the skin or it may manifest as systemic LE and affect any organ system in the body, most commonly the kidneys, joints and blood.

Cutaneous lupus is usually treated by using anti-malarials and corticosteroids. However, these drugs may not be effective for treating some cutaneous lupus or they may have serious side effects when they are continuously used for a long period of time. Therefore, it has been desired to develop new therapeutic methods of treating cutaneous lupus.

### 3. SUMMARY OF THE INVENTION

In one aspect, the invention provides methods of treating, preventing and/or managing cutaneous lupus in humans including, but not limited to, acute cutaneous lupus erythematosus (ACLE), subacute cutaneous lupus erythematosus (SCLE), neonatal lupus erythematosus (NLE), lupus erythematosus of childhood, and chronic cutaneous lupus erythematosus (CCLE) or discoid lupus erythematosus (DLE) (e.g., verrucous DLE, lupus profundus, mucosal DLE, palmar-plantar DLE and lupus tumidus). The invention provides methods of treating, preventing and/or managing cutaneous lupus in human including, but not limited to, men, women, and children.

In one aspect, the methods comprise administering to a patient in need of such treatment, prevention or management a therapeutically or prophylactically effective amount of (+)-2-[1-(3-ethoxy-4-methoxyphenyl)-2-methylsulfonylethyl]-4-acetylaminoisoindoline-1,3-dione, or a pharmaceutically acceptable salt or solvate (e.g., hydrate) thereof, substantially free of its (−) enantiomer. In a preferred embodiment, a salt or solvate of the compound is used if not the free compound.

In one aspect, the invention provides methods which comprise administering to a patient in need of such treatment, prevention or management a therapeutically or prophylactically effective amount of 4-(amino)-2-(2,6-dioxo(3-piperidyl))-isoindoline-1,3-dione, or a pharmaceutically acceptable prodrug, metabolite, polymorph, salt, solvate (e.g., hydrate), stereoisomer or clathrate thereof. In a preferred embodiment, a salt or solvate of the compound is used.

In one aspect, the invention provides methods which comprise administering to a patient in need of such treatment, prevention or management a therapeutically or prophylactically effective amount of 3-(4 amino-1-oxo-1,3-dihydro-isoindol-2-yl)-piperidine-2,6-dione, or a pharmaceutically acceptable prodrug, metabolite, polymorph, salt, solvate (e.g., hydrate), stereoisomer or clathrate thereof. In a preferred embodiment, a salt or solvate of the compound is used.

In one aspect, the invention provides methods of treating, preventing and/or managing cutaneous lupus with cyclopropyl {2-[(1S)-1-(3-ethoxy-4-methoxyphenyl)-2-(methylsulfonyl)ethyl]-3-oxoisoindolin-4-yl}carboxamide, or a pharmaceutically acceptable salt or solvate (e.g., hydrate) thereof, substantially free of its (R)-enantiomer. In other embodiments, a salt or solvate of the compound is used if not the free compound.

In some embodiments, the methods further comprise the administration of a therapeutically effective amount of at least a second active agent which may be an anti-inflammatory such as non-steroidal agents (e.g., salicylates) or corticosteroids (e.g., dexamethasone), an anti-malarial, an immunosuppressant, an antibiotic, an antiviral, an immunologic-enhancing drug, a hormone, PGE2 or a combination thereof.

In another embodiment, the compounds of the invention or a pharmaceutically acceptable salt, solvate or stereoisomer thereof are administered topically in a dosage form which includes, but is not limited to, ointments, creams, gels, pastes, dusting powders, lotions, sprays, liniments, poultices, aerosols, solutions, emulsions, suspensions and combinations thereof.

In further embodiments, the compounds of the invention or a pharmaceutically acceptable salt, solvate or stereoisomer thereof are administered parenterally or orally or in a controlled-release manner.

### 4. BRIEF DESCRIPTION OF THE FIGURES

FIG. 1 illustrates the cell expression of CD51/61 and ICAM-1 on HUVEC in unstimulated conditions.

FIG. 2 illustrates the cell expression of E-Selectin and P-Selectin on HUVEC in unstimulated conditions.

FIG. 3 illustrates the cell expression of E-Selectin and P-Selectin on HUVEC in TNF-α-stimulated conditions.

FIG. 4 illustrates the cell expression of VE-cadherin and CD44 on HUVEC in TNF-α-stimulated conditions.

FIG. 5 illustrates the cell expression of CD51/61, ICAM-1, ICAM-2, VCAM-1, E-Selectin, P-Selectin, HLA Class I and HLA Class II on HUVEC in TNF-α-stimulated conditions.

FIG. 6 illustrates the cell expression of E-Selectin on HUVEC in TNF-α-stimulated conditions where E-Selectin was detected by ELISA.

FIG. 7 illustrates study for ultraviolet B-induced TNF-alpha production by human keratinocytes.

FIG. 8 illustrates study for ultraviolet B-induced TNF-alpha production by human keratinocytes.

FIG. 9 illustrates study for ultraviolet B-induced TNF-alpha production by human keratinocytes.

### 5. DETAILED DESCRIPTION OF THE INVENTION

One aspect of the invention encompasses methods of treating, managing and/or preventing cutaneous lupus which comprise administering to a patient having cutaneous lupus a therapeutically or prophylactically effective amount of (+)-2-[1-(3-ethoxy-4-methoxyphenyl)-2-methylsulfonylethyl]-4-acetylaminoisoindoline-1,3-dione, or a pharmaceutically acceptable salt or solvate thereof, substantially free of its (−) enantiomer.

Another aspect of the invention encompasses methods of treating, managing and/or preventing cutaneous lupus which comprise administering to a patient having cutaneous lupus a therapeutically or prophylactically effective amount of 4-(amino)-2-(2,6-dioxo(3-piperidyl))-isoindoline-1,3-dione, or a pharmaceutically acceptable prodrug, metabolite, polymorph, salt, solvate (e.g., hydrate), stereoisomer or clathrate thereof.

Another aspect of the invention encompasses methods of treating, managing and/or preventing cutaneous lupus which comprise administering to a patient having cutaneous lupus a therapeutically or prophylactically effective amount of 3-(4 amino-1-oxo-1,3-dihydro-isoindol-2-yl)-piperidine-2,6-dione, or a pharmaceutically acceptable prodrug, metabolite, polymorph, salt, solvate (e.g., hydrate), stereoisomer or clathrate thereof.

Another aspect of the invention encompasses methods of treating, managing and/or preventing cutaneous lupus which comprise administering to a patient having cutaneous lupus a therapeutically or prophylactically effective amount of cyclopropyl {2-[1S)-1-(3-ethoxy-4-methoxyphenyl)-2-(methylsulfonyl)ethyl]-3-oxoisoindolin-4-yl}carboxamide, or a pharmaceutically acceptable salt or solvate thereof, substantially free of its (R)-enantiomer.

Examples of cutaneous lupus within the scope of the present invention include, but not limited to, acute cutaneous lupus erythematosus (ACLE), subacute cutaneous lupus erythematosus (SCLE), neonatal lupus erythematosus (NLE), lupus erythematosus of childhood and discoid lupus erythematosus (DLE) including verrucous DLE, lupus profundus, mucosal DLE, palmar-plantar DLE and lupus tumidus.

Furthermore, the patients to be treated included mammals, particularly human. Children and adults can be treated by the methods and compositions disclosed herein. Immunocompromised patients may also be treated. This invention contemplates treatment of patients that have not used other therapies, those that have used other therapies and those refractory to therapies for lupus such as cutaneous lupus mentioned above. In some embodiments, the patient is a female. In some embodiments, the patient is a male. In further embodiments, the patient is a child.

### 5.1 Definitions

As used herein and unless otherwise indicated, the term “the compound of the invention” includes, but is not limited to, (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl {2-[(1S)-1-(3-ethoxy-4-methoxyphenyl)-2-(methylsulfonyl)ethyl]-3-oxoisoindolin-4-yl}carboxamide, or a pharmaceutically acceptable prodrug, metabolite, polymorph, salt, solvate, stereoisomer or clathrate thereof.

As used herein and unless otherwise indicated, the term “pharmaceutically acceptable salt” includes, but is not limited to, salts of acidic or basic groups that can be present in the compounds of the invention. Under certain acidic conditions, the compound of the invention can form a wide variety of salts with various inorganic and organic acids. The acids that can be used to prepare pharmaceutically acceptable salts of such basic compounds are those that form salts comprising pharmacologically acceptable anions including, but not limited to, acetate, benzenesulfonate, benzoate, bicarbonate, bitartrate, bromide, calcium edetate, camsylate, carbonate, chloride, bromide, iodide, citrate, dihydrochloride, edetate, edisylate, estolate, esylate, fumarate, gluceptate, gluconate, glutamate, glycollylarsanilate, hexylresorcinate, hydrabamine, hydroxynaphthoate, isethionate, lactate, lactobionate, malate, maleate, mandelate, mesylate, methylsulfate, muscate, napsylate, nitrate, panthothenate, phosphate/diphosphate, polygalacturonate, salicylate, stearate, succinate, sulfate, tannate, tartrate, teoclate, triethiodide and pamoate. Under certain basic conditions, the compound of the invention can form base salts with various pharmacologically acceptable cations. Non-limiting examples of such salts include alkali metal or alkaline earth metal salts and, particularly, calcium, magnesium, sodium, lithium, zinc, potassium and iron salts.

As used herein and unless otherwise indicated, the term “hydrate” means a compound of the present invention or a salt thereof, that further includes a stoichiometric or non-stoichiometric amount of water bound by non-covalent intermolecular forces.

As used herein and unless otherwise indicated, the term “solvate” means a solvate formed from the association of one or more solvent molecules to a compound of the present invention. The term “solvate” includes hydrates (e.g., mono-hydrate, dihydrate, trihydrate, tetrahydrate and the like).

As used herein and unless otherwise indicated, the term “polymorph” means solid crystalline forms of a compound of the present invention or complex thereof. Different polymorphs of the same compound can exhibit different physical, chemical and/or spectroscopic properties.

As used herein and unless otherwise specified, the term “prodrug” means a derivative of a compound that can hydrolyze, oxidize, or otherwise react under biological conditions (in vitro or in vivo) to provide the compound. Examples of prodrugs include, but are not limited to, derivatives and metabolites of (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl {2-[(1S)-1-(3-ethoxy-4-methoxyphenyl)-2-(methylsulfonyl)ethyl]-3-oxoisoindolin-4-yl}carboxamide that include biohydrolyzable moieties such as biohydrolyzable amides, biohydrolyzable esters, biohydrolyzable carbamates, biohydrolyzable carbonates, biohydrolyzable ureides, and biohydrolyzable phosphate analogues. Prodrugs can typically be prepared using well-known methods, such as those described by 1 Burger's Medicinal Chemistry and Drug Discovery, 172-178, 949-982 (Manfred E. Wolff ed., 5th ed. 1995).

As used herein, and unless otherwise specified, the terms “biohydrolyzable carbamate,” “biohydrolyzable carbonate,” “biohydrolyzable ureide” and “biohydrolyzable phosphate” mean a carbamate, carbonate, ureide and phosphate, respectively, of a compound that either: 1) does not interfere with the biological activity of the compound but can confer upon that compound advantageous properties in vivo, such as uptake, duration of action or onset of action; or 2) is biologically inactive but is converted in vivo to the biologically active compound. Non-limiting examples of biohydrolyzable carbamates include lower alkylamines, substituted ethylenediamines, aminoacids, hydroxyalkylamines, heterocyclic and heteroaromatic amines and polyether amines.

As used herein, and unless otherwise specified, the term “stereoisomer” encompasses all enantiomerically/stereomerically pure and enantiomerically/stereomerically enriched compounds of this invention.

As used herein, and unless otherwise indicated, the term “stereomerically pure” or “enantiomerically pure” means that a compound comprises one stereoisomer and is substantially free of its counter stereoisomer or enantiomer. For example, a compound is stereomerically or enantiomerically pure when the compound contains 80%, 90% or 95% or more of one stereoisomer and 20%, 10% or 5% or less of the counter stereoisomer. In some cases, a compound of the invention is considered optically active or stereomerically/enantiomerically pure (e.g., substantially the R-form or substantially the S-form) with respect to a chiral center when the compound is about 80% ee (enantiomeric excess) or greater, preferably, equal to or greater than 90% ee with respect to a particular chiral center and more preferably 95% ee with respect to a particular chiral center.

As used herein, and unless otherwise indicated, the term “substantially free of its (R)-enantiomer” is used herein to mean equal to or greater than 80% pure of the (S)-enantiomer, based upon the total weight of the compound. In some instances, the term “substantially free of its (R)-enantiomer” means equal to or greater than 85%, 90%, 95% or 99% pure of the (S)-enantiomer, based upon the total weight of the compound.

As used herein, and unless otherwise indicated, the term “substantially free of its (−) enantiomer” is used herein to mean equal to or greater than 80% pure of the (+) enantiomer, based upon the total weight of the compound. In some instances, the term “substantially free of its (−) enantiomer” means equal to or greater than 85%, 90%, 95% or 99% pure of the (+) enantiomer, based upon the total weight of the compound.

As used herein, and unless otherwise indicated, the term “stereomerically enriched” or “enantiomerically enriched” encompasses certain mixtures of stereoisomers of compounds of this invention (e.g., R/S=30/70, 35/65, 65/35 and 70/30).

As used herein, and unless otherwise specified, the terms “treat,” “treating” and “treatment” contemplate an action that occurs while a patient is suffering from the specified disease or disorder, which reduces the severity or symptoms of the disease or disorder or retards or slows the progression or symptoms of the disease or disorder.

As used herein, and unless otherwise specified, the term “therapeutically effective amount” encompasses the above described dosage amounts and dose frequency schedules. Different therapeutically effective amounts may be applicable for different lupus disorders and conditions, as will be readily known by those of ordinary skill in the art. Similarly, amounts sufficient to treat or prevent such disorders, but insufficient to cause, or sufficient to reduce, adverse effects associated with the compounds of the invention are also encompassed by the above described dosage amounts and dose frequency schedules.

As used herein, unless otherwise specified, the terms “prevent,” “preventing” and “prevention” contemplate an action that occurs before a patient begins to suffer from the specified disease or disorder, which inhibits or reduces the severity or symptoms of the disease or disorder.

As used herein, and unless otherwise indicated, the terms “manage,” “managing” and “management” encompass preventing the recurrence of the specified disease or disorder in a patient who has already suffered from the disease or disorder and/or lengthening the time that a patient who has suffered from the disease or disorder remains in remission. The terms encompass modulating the threshold, development and/or duration of the disease or disorder or changing the way that a patient responds to the disease or disorder.

As used herein, and unless otherwise specified, the term “enhancing” or “enhance,” when used in connection with immune response, means that when an antigenic or immunogenic agent is administered to a subject who has been or is being treated with the compounds of the invention, there is an increased antibody formation, as compared to a subject to which same amount of the antigenic or immunogenic agent alone is administered, as determined by any conventional methods of antibody level determination known in the art, for example, nephelometry, immunoelectrophoresis, radioimmunoassay and ELISA. In some embodiments, when methods of this invention are used, antibody formation is increased by about 5%, 10%, 20%, 50% or 100% or more, as compared to the antibody formation obtained when such methods are not used.

### 5.2 The Compound of the Invention

### (+)-2-[1-(3-Ethoxy-4-methoxyphenyl)-2-mehtyl sulfonylethyl]-4-acetylaminoisoindoline-1,3-dione

The present invention provides methods of treating, managing or preventing cutaneous lupus, which comprises administering to a patient in need of such treatment, management or prevention a therapeutically or prophylactically effective amount of (+) enantiomer of (+)-2-[1-(3-ethoxy-4-methoxyphenyl)-2-methylsulfonylethyl]-4-acetylaminoisoindoline-1,3-dione.

Without being limited by theory, the (+) enantiomer of (+)-2-[1-(3-ethoxy-4-methoxyphenyl)-2-methylsulfonylethyl]-4-acetylaminoisoindoline-1,3-dione is believed to be (S)-{2-[1-(3-ethoxy-4-methoxyphenyl)-2-methylsulfonylethyl]-4-acetylaminoisoindoline-1,3-dione} [Compound (I)], which has the following structure:

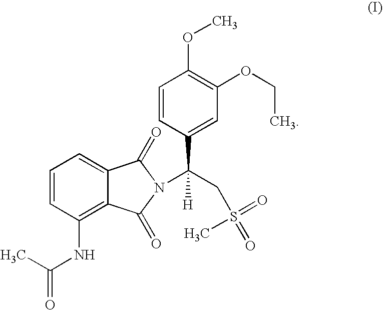

Thus, (+)-2-[1-(3-ethoxy-4-methoxyphenyl)-2-methylsulfonylethyl]-4-acetylaminoisoindoline-1,3-dione is used to describe the compound depicted as Compound (I). Compound (I) can be prepared according to methods disclosed in U.S. Pat. No. 6,962,940, titled “(+)-2-[1-(3-Ethoxy-4-methoxyphenyl)-2-methylsulfonylethyl]-4-acetylaminoisoindoline-1,3-dione: Methods Of Using And Compositions Thereof,” issued Nov. 8, 2005, which is incorporated herein by reference. In a specific method, Compound (I) is synthesized from 3-acetamidophthalic anhydride and a chiral amino acid salt of (S)-2-(3-ethoxy-4-methoxyphenyl)-1-(methylsulphonyl)-eth-2-ylamine. Chiral amino acid salts of (S)-2-(3ethoxy-4-methoxyphenyl)-1-(methylsulphonyl)-eth-2-ylamine include, but not limited to salts formed with the L isomers of alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine, ornithine, 4-aminobutyric acid, 2-aminoisobutyric acid, 3-aminopropionic acid, ornithine, norleucine, norvaline, hydroxyproline, sarcosine, citrulline, cysteic acid, t-butylglycine, t-butylalanine, phenylglycine, cyclohexylalanine, and N-acetyl-L-leucine. A specific chiral amino acid salt is (S)-2-(3-ethoxy-4-methoxyphenyl)-1-(methylsulphonyl)-eth-2-ylamine N-acetyl-L-leucine salt, which is resolved from 2-(3-ethoxy-4-methoxyphenyl)-1-(methylsulphonyl)-eth-2-ylamine and N-acetyl-L-leucine in methanol.

Alternatively, Compound (I) can be isolated from the corresponding racemic 2-[1-(3-ethoxy-4-methoxyphenyl)-2-methylsulfonylethyl]-4-acetylaminoisoindoline-1,3-dione by separation techniques known in the art. The racemic compound can be readily prepared according to the procedure for Example 12 of U.S. Pat. No. 6,020,358, which is incorporated herein by reference. Examples of suitable separation techniques include, but are not limited to, the formation of chiral salts and the use of chiral or high performance liquid chromatography “HPLC” and the formation and crystallization of chiral salts. See, e.g., Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

### 4-(Amino)-2-(2,6-dioxo(3-piperidyl))-isoindoline-1,3-dione and 3-(4-amino-1-oxo-1,3-dihydro-isoindol-2-yl)-piperidine-2,6-Dione

The present invention provides methods of treating, managing or preventing cutaneous lupus, which comprise administering to a patient in need of such treatment, management or prevention a therapeutically or prophylactically effective amount of 4-(amino)-2-(2,6-dioxo(3-piperidyl))-isoindoline-1,3-dione (ACTIMID™) having the following formula:

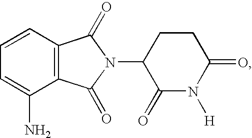

or 3-(4-amino-1-oxo-1,3-dihydro-isoindol-2-yl)-piperidine-2,6-dione (REVLIMID®) having the following chemical structure: 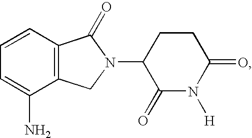
or a pharmaceutically acceptable prodrug, metabolite, polymorph, salt, solvate, stereoisomer or clathrate thereof.

The compounds are available from Celgene Corporation, Summit, N.J. The compounds can be obtained via standard, synthetic methods (see e.g., U.S. Pat. No. 5,635,517, incorporated herein by reference). The specific methods of preparing 4-(amino)-2-(2,6-dioxo(3-piperidyl))-isoindoline-1,3-dione are disclosed in U.S. Patent Non-Provisional Application No. 11/479,823 filed on Jun. 29, 2006 and U.S. Patent Provisional Application 60/696,224 filed on Jun. 30, 2005, titled “Processes for the preparation of 4-amino-2-(2,6-dioxopiperidin-3-yl)-isoindoline-1,3-dione compounds,” all of which are incorporated herein by reference.

In one embodiment, 4-(amino)-2-(2,6-dioxo(3-piperidyl))-isoindoline-1,3-dione or 3-(4-amino-1-oxo-1,3-dihydro-isoindol-2-yl)-piperidine-2,6-dione is enantiomerically pure. In a further embodiment, 4-(amino)-2-(2,6-dioxo(3-piperidyl))-isoindoline-1,3-dione or 3-(4-amino-1-oxo-1,3-dihydro-isoindol-2-yl)-piperidine-2,6-dione is the R-enantiomer. In a further embodiment, 4-(amino)-2-(2,6-dioxo(3-piperidyl))-isoindoline-1,3-dione or 3-(4-amino-1-oxo-1,3-dihydro-isoindol-2-yl)-piperidine-2,6-dione is the S-enantiomer. In a further embodiment, 4-(amino)-2-(2,6-dioxo(3-piperidyl))-isoindoline-1,3-dione or 3-(4-amino-1-oxo-1,3-dihydro-isoindol-2-yl)-piperidine-2,6-dione is a racemic mixture.

In further embodiments, specific compounds used in the invention are polymorphic forms of 4-(amino)-2-(2,6-dioxo(3-piperidyl))-isoindoline-1,3-dione or 3-(4-amino-1-oxo-1,3-dihydro-isoindol-2-yl)-piperidene-2,6-dione. Specific polymorphic forms of 3-(4-amino-1-oxo-1,3-dihydro-isoindol-2-yl)-piperidene-2,6-dione, such as Form A, B, C, D, E, F, G and H, are disclosed in U.S. provisional application No. 60/499,723 filed on Sep. 4, 2003, and U.S. non-provisional application No. 10/934,863 (publication no. 2005/0096351) filed on Sep. 3, 2004, which are incorporated herein by reference in their entireties.

For example, Form A of 3-(4-amino-1-oxo-1,3-dihydro-isoindol-2-yl)-piperidene-2,6-dione is an unsolvated, crystalline material that can be obtained from non-aqueous solvent systems. Form A has an X-ray powder diffraction pattern comprising significant peaks at approximately 8, 14.5, 16, 17.5, 20.5, 24 and 26 degrees 2θ, and has a differential scanning calorimetry melting temperature maximum of about 270° C. Form B of 3-(4-amino-1-oxo-1,3-dihydro-isoindol-2-yl)-piperidene-2,6-dione is a hemihydrated, crystalline material that can be obtained from various solvent systems, including, but not limited to, hexane, toluene, and water. Form B has an X-ray powder diffraction pattern comprising significant peaks at approximately 16, 18, 22 and 27 degrees 2θ, and has a differential scanning calorimetry melting temperature maximum of about 268° C.

### Cyclopropyl {2-[(1S)-1-(3-ethoxy-4-methoxyphenyl)-2-(methylsulfonyl)ethyl]-3-oxoisoindolin-4-yl}carboxamide

The present invention provides methods of treating, managing or preventing cutaneous lupus, which comprises administering to a patient in need of such treatment, management or prevention a therapeutically or prophylactically effective amount of cyclopropyl {2-[(1S)-1-(3-ethoxy-4-methoxyphenyl)-2-(methylsulfonyl)ethyl]-3-oxoisoindolin-4-yl}carboxamide or N-[2-[(1S)-1-(3-ethoxy-4-methoxyphenyl)-2-(methylsulfonyl)ethyl]-2,3-dihydro-3-oxo-1H-isoindol-4-yl]-cyclopropanecarboxamide.

Cyclopropyl {2-[(1S)-1-(3-ethoxy-4-methoxyphenyl)-2-(methylsulfonyl)ethyl]-3-oxoisoindolin-4-yl}carboxamide or N-[2-[(1S)-1-(3-ethoxy-4-methoxyphenyl)-2-(methylsulfonyl)ethyl]-2,3-dihydro-3-oxo-1H-isoindol-4-yl]-cyclopropanecarboxamide [i.e., Compound (II)] has the following structure:

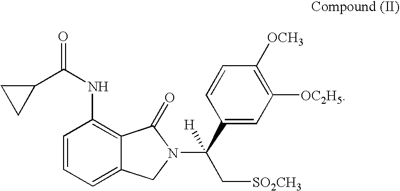

Compound (II) can be prepared according to the preparation procedure for Example 57 of U.S. Pat. No. 6,667,316, titled “Pharmaceutically Active Isoindoline Derivatives,” issued Dec. 23, 2003, which is incorporated herein by reference in its entirety. In a specific embodiment, Compound (II) can be prepared by heating a mixture of 7-amino-2-[(1S)-1-(3-ethoxy-4-methoxyphenyl)-2-(methylsulfonyl)ethyl]isoindolin-1-one and cyclopropanecarbonyl chloride in tetrahydrofuran.

Alternatively, Compound (II) can be isolated from the corresponding racemic cyclopropyl {2-[1-(3-ethoxy-4-methoxyphenyl)-2-(methylsulfonyl)ethyl]-3-oxoisoindolin-4-yl}carboxamide by separation techniques known to skilled artisans. The racemic compound can be readily prepared according to the preparation procedure for Example 55 of U.S. Pat. No. 6,667,316. Examples of suitable separation techniques include, but are not limited to, the formation of chiral salts and the use of chiral or high performance liquid chromatography “HPLC” and the formation and crystallization of chiral salts. See, e.g., Rex W. Souter, Chromatographic Separations of Stereoisomers, (CRC Press, Boca Raton, 1985); Jacques, J., et al., Enantiomers, Racemates and Resolutions (Wiley Interscience, New York, 1981); Wilen, S. H., et al., Tetrahedron 33:2725 (1977); Eliel, E. L., Stereochemistry of Carbon Compounds (McGraw Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

### 5.3 Methods of Treatments and Prevention

The present invention provides methods of treating, preventing and/or managing cutaneous lupus. Non-limiting examples of cutaneous lupus within the scope of the method of the invention include, but are not limited to, cutaneous lupus erythematosus (CLE), acute cutaneous lupus erythematosus (ACLE), subacute cutaneous lupus erythematosus (SCLE), chronic cutaneous lupus erythematosus (CCLE) or discoid lupus erythematosus (DLE), neonatal lupus erythematosus (NLE), verrucous DLE, lupus profundus, mucosal DLE, palmar-plantar DLE and lupus tumidus.

In some embodiments, the present invention provides methods of treating ACLE. ACLE is generally a photosensitive dermatosis. It can appear as flattened areas of red skin that resemble a persistent sunburn or have a rash-like appearance. ACLE may erupt in a butterfly pattern localized to the central portion of the face and/or in a generalized pattern including other areas such as the arms, legs and body. The etiology of ACLE is believed to be multi-factorial, involving genetic, environmental and hormonal factors. Thus, the invention includes treatment in patients who are predisposed genetically or exposed to natural ultraviolet radiation.

In further embodiments, the present invention provides methods of treating SCLE. SCLE is a non-scarring non-atrophy-producing photosensitive dermatosis. In some cases, SCLE appears as a non-itchy ring-shaped dry rash on the upper back and chest, often following sun exposure. SCLE may occur in patients with systemic lupus erythematosus, Sjögren syndrome and deficiency of the second component of complement (C2d) or it can be drug induced. SCLE usually occurs in genetically predisposed individuals, most often in patients with human leukocyte antigen B8 (HLA-B8), human leukocyte antigen DR3 (HLA-DR3), human leukocyte antigen DRw52 (HLA-DRw52) and human leukocyte antigen DQ1 (HLA-DQ1). SCLE strongly associates with anti-Ro (SS-A) autoantibodies. Thus, in a particular embodiment, the invention includes treatment of such patient population.

In further embodiments, the present invention provides methods of treating CCLE or DLE. CCLE or DLE is a chronic, scarring, atrophy producing, photosensitive dermatosis. DLE commonly appears as red scaly patches which leave white scars. DLE predominantly affects the cheeks and nose, but sometimes involves the upper back, neck, backs of hands, bald areas in scalp and the lips. DLE may occur in patients with systemic lupus erythematosus (SLE). Some patients also have the lesions of SCLE and some may have a malar rash. DLE occurs in genetically predisposed individuals. Thus, in a particular embodiment, the invention includes treatment of such patient population.

In further embodiments, the present invention provides methods of treating verrucous DLE in a human via oral or topical administration. Verrucous DLE is a specific form of DLE and refers to DLE having lesions that can develop into very thick scales.

In further embodiments, the present invention provides methods of treating lupus profundus in a human via oral or topical administration. Lupus profundus is a specific form of DLE and refers to DLE having lesions that may occur in conjunction with firm lumps in the fatty tissue underlying the skin.

In further embodiments, the present invention provides methods of treating mucosal DLE in a human via oral or topical administration. Mucosal DLE is a specific form of DLE and refers to the lesions that occasionally occur in the mucus membranes of the mouth, nose and eyes.

In further embodiments, the present invention provides methods of treating palmar-plantar DLE in a human via oral or topical administration. Palmar-plantar DLE is a specific form of DLE and refers to the lesions that occasionally occur on the hands and feet.

In further embodiments, the present invention provides methods of treating lupus tumidus in a human via oral or topical administration. Lupus tumidus is a specific form of DLE and appears as smooth, shiny, red-violet plaques of the head and neck that can be pruritic and have a fine scale. The lupus tumidus lesions usually clear without scarring and can recur in their original distribution.

In further embodiments, the present invention provides methods of treating NLE. NLE is a rare condition in children and usually appears as nonscarring, non-atrophy-producing lesions. In particular embodiments, the methods include oral or topical or both treatment of newborn babies born to mothers with SCLE. NLE is believed to be related to various factors including genetic predisposition, viral infection and other unknown factors.

In further embodiments, the present invention provides methods of treating Lupus erythematosus (LE) of childhood. In a particular embodiment, lupus erythematosus (LE) is treated in children including children predisposed to genetic factors and perhaps other environmental events.

This invention also encompasses the uses of the compounds of the invention in modulating the immune system to keep it from slipping into imbalance and producing inflammatory and autoimmune disorders like lupus in a patient. Therefore, in another embodiment, this invention encompasses methods of enhancing an immune response to an immunogen, comprising administering a therapeutically or prophylactically effective amount of (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl 2-[(1S)-1-(3-ethoxy-4-methoxyphenyl)-2-(methylsulfonyl)ethyl]-3-oxoisoindolin-4-yl}carboxamide, or a pharmaceutically acceptable salt, solvate or stereoisomer thereof, to a patient in need of such enhancement. The compounds can be administered prior to, during or subsequent to the patient's exposure to the immunogen.

5.3.1 Combination Therapy with a Second Active Agent

In particular methods encompassed by this embodiment, the compound of the invention is administered in combination with another drug (“second active agent”) in methods of treating, managing and/or preventing cutaneous lupus. The second active agent includes, but is not limited to, anti-inflammatory agents such as non-steroidal agents and corticosteroids, anti-malarials, immunosuppressants, antibiotics, antivirals, immunologic-enhancing drugs, hormones, PGE2 and combinations thereof. Non-limiting examples of methods or therapies that can be used in combination with the administration of the compound of the invention include antibody injections or infusions, and stem cell transplantation.

The compound of the invention can be used with at least a second active agent in methods of the invention disclosed herein. This invention encompasses synergistic combinations for the treatment, prevention and/or management of cutaneous lupus. The compound of the invention can also be used to alleviate adverse or unnamed effects associated with some second active agents, and conversely some second active agents can be used to alleviate adverse or unnamed effects associated with the compound of the invention.

In some embodiments of interest, the second active agents may include, but are not limited to, anti-inflammatories such as, but not limited to, acetaminophen (e.g., TYLENOL®), 5-aminosalicylic acid derivatives, salicylates, corticosteroids and nonsteroidal anti-inflammatory drugs. A non-limiting example of 5-aminosalicylic acid derivatives is sulfasalazine (e.g., AZULFIDINE®). A non-limiting examples of salicylates is acetylsalicylic acid (e.g., ASPIRIN®).

Non-limiting examples of corticosteroids include dexamethasone (e.g., AZIUM® or VOREN®), hydrocortisone (e.g., CETACORT®, HYTONE® or NUTRACORT®), beclomethasone (e.g. , VANCERIL®), budesonide (e.g., PULMICORT®), fluticasone (e.g., FLONASE® or FLOVENT®), methylprednisolone (e.g., DEPO-MEDROL®, SOLU-MEDROL® or MEDROL®), mometasone furoate (e.g., NASONE® or ELOCON®), prednisone (e.g., DELTASON®, ORASON®, PREDNICEN-M® or LIQUID PRED®) and triamcinolone (e.g., AZMACORT®).

Non-limiting examples of nonsteroidal anti-inflammatory drugs include diclofenac (e.g., ARTHROTEC®), diflunisal (e.g., DOLOBID®), etodolac (e.g., LODINE®) fenoprofen (e.g., NALFON®), ibuprofen (e.g., ADVIL®, CHILDREN'S ADVIL/MOTRIN®, MEDIPREN®, MOTRIN®, NUPRIN® or PEDIACARE FEVER®), indomethacin (e.g., ARTHREXIN®), ketoprofen (e.g., ORUVAIL®), ketorolac (e.g., TORADOL®), fosfomycin tromethamine (e.g., MONURAL®), meclofenamate (e.g., Meclomen®), nabumetone (e.g., RELAFEN®), naproxen (e.g., ANAPROX®, ANAPROX® DS, EC-NAPROSYN®, NAPRELAN® or NAPROSYN®), oxaprozin (e.g., DAYPRO®), piroxicam (e.g., FELDENE®), sulindac (e.g., CLINORIL®), and tolmetin (e.g., TOLECTIN® DS or TOLECTIN®).

In other embodiments of interest, the second active agents may include, but are not limited to, anti-malarials such as chloroquine (e.g., ARALEN®) and hydroxychloroquine (e.g., PLAQUENIL®); immunosuppressants such as azathioprine (e.g., IMURAN®^{M}), cyclophosphamide (e.g., CYTOXAN®), chlorambucil (e.g., LEUKERAN®) and melphalan (e.g., ALKERAN®); and immunomodulatory compounds such as azathioprine (e.g., IMURAN®), cyclophosphamide (e.g., CYTOXAN®), methotrexate (e.g., RHEUMATREX®) and cyclosporin (e.g., NEORAL® or SANDIMMUNE®).

In further embodiments of interest, the second active agents may include, but are not limited to, antibiotics (therapeutic or prophylactic) such as, but not limited to, ampicillin (e.g., UNASYN®), tetracycline (e.g., ACHROMYCIN® or SUMYCIN®), penicillin (e.g., AMOXIL®, POLYMOX®, TRIMOX®, SPECTROBID® or GEOCILLIN®), cephalosporins (e.g., OMNICEF®, SPECTRACEF®, SUPRAX®, VANTIN®, CEFZIL® or CEDAX®), streptomycin (e.g., ZANOSAR®), kanamycin (e.g., KANTREX®) and erythromycin (e.g., E.E.S.®, E-MYCIN®, ERYC®, ERY-TAB®, ERYTHROCIN® or PCE®); antivirals such as, but not limited to, amantadine (e.g., SYMMETREL®), rimantadine (e.g., FLUMADINE®), acyclovir (e.g., ZOVIRAX®) and ribavirin (e.g., VIRAZOLE®); immunoglobulin; immunologic enhancing drugs such as, but not limited to, levamisole (e.g., ERGAMISOL®) and inosine pranobex (ISOPRINOSINE®); biologics such as, but not limited to, gammaglobulin, transfer factor, interleukins and interferons; hormones such as, but not limited to, thymic; and other immunologic agents such as, but not limited to, B cell stimulators (e.g., BAFF/BlyS), cytokines (e.g., IL-2, IL-4 and IL-5), growth factors (e.g., TGF-β), antibodies (e.g., anti-CD40 and IgM), oligonucleotides containing unmethylated CpG motifs (e.g., TCGTCGTTTTGTCGTTTTGTCGTT) and vaccines (e.g., viral and tumor peptide vaccines).

In another embodiment, methods of this invention can be used in combination with other methods used for the treatment, prevention and/or management of cutaneous lupus. Examples of other methods include, but not limited to, stem cell transplantation, enzyme replacement therapy using, for example, bovine adenosine deaminase conjugated to polyethylene glycol (PEG-ADA), fetal thymus transplant, cultured neonatal thymus transplant, thymic epithelial cell transplant and fetal liver transplant.

Specific methods of the invention comprise administering (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl 2-[(1S)-1-(3-ethoxy-4-methoxyphenyl)-2-(methylsulfonyl)ethyl]-3-oxoisoindolin-4-yl}carboxamide, or a pharmaceutically acceptable salt, solvate or stereoisomer thereof, in combination with at least a second active agent or another therapy.

Administration of the compound of the invention and at least a second active agent to a patient can occur simultaneously or sequentially by the same or different routes of administration. The suitability of a particular route of administration employed for a particular second active agent will depend on the second active agent itself (e.g., whether it can be administered topically or orally without decomposition prior to entering the blood stream) and the disease being treated. A particular route of administration for the compound of the invention is topical administration. Particular routes of administration for the second active agents or ingredients of the invention are known to those of ordinary skill in the art. See, e.g., The Merck Manual, 430-431 (17^{th} ed., 1999).

The amount of second active agent administered can be determined based on the specific agent used, the type of disease being treated or managed, the severity and stage of disease and the amount(s) of the compounds of the invention and any optional additional second active agents concurrently administered to the patient. Those of ordinary skill in the art can determine the specific amounts according to conventional procedures known in the art. In the beginning, one can start from the amount of the second active agent that is conventionally used in the therapies and adjust the amount according to the factors described above. See, e.g., Physician's Desk Reference (56^{th} Ed., 2004). Further, the amounts and methods of administration of the second active agents disclosed herein for the treatment, prevention and/or management of cutaneous lupus are disclosed in the literature, e.g., Physician's Desk Reference (56^{th} Ed., 2004), which is incorporated herein by reference.

5.3.2 Cycling Therapy

In some embodiments, the compound of the invention can be cyclically administered to a patient. Cycling therapy involves the administration of the compound of the invention for a period of time, followed by a rest for a period of time and repeating this sequential administration. Cycling therapy can reduce the development of resistance to one or more of the therapies, avoid or reduce the side effects of one of the therapies and/or improves the efficacy of the treatment.

Consequently, in one specific embodiment of the invention, the compound of the invention is administered daily in a single or divided doses in a four to six week cycle with a rest period of about a week or two weeks. The invention further allows the frequency, number and length of dosing cycles to be increased. Thus, another specific embodiment of the invention encompasses the administration of the compound of the invention for more cycles than are typical when it is administered alone. In yet another specific embodiment of the invention, the compound of the invention is administered for a greater number of cycles that would typically cause dose-limiting toxicity in a patient to whom a second active ingredient is not also being administered.

In one embodiment, (+)-2-[1-(3-ethoxy-4-methoxyphenyl)-2-methylsulfonylethyl]-4-acetylaminoisoindoline-1,3-dione is administered daily and continuously for three or four weeks at a dose of from about 10 to about 200 mg per day followed by a break of one or two weeks. In another embodiment, 4-(amino)-2-(2,6-dioxo(3-piperidyl))-isoindoline-1,3-dione is administered daily and continuously for three or four weeks at a dose of from about 0.1 to 5 mg per day followed by a break of one or two weeks. In a particular embodiment, 3-(4-amino-1-oxo-1,3-dihydro-isoindol-2-yl)-piperidine-2,6-dione is administered in an amount of about 5, 10, 25 or 50 mg/day, preferably in an amount of about 25 mg/day for three to four weeks, followed by one or two weeks of rest in a four or six week cycle. In another embodiment, cyclopropyl {2-[(1S)-1-(3-ethoxy-4-methoxyphenyl)-2-(methylsulfonyl)ethyl]-3-oxoisoindolin-4-yl}carboxamide is administered daily and continuously for three or four weeks at a dose of from about 10 to about 200 mg per day followed by a break of one or two weeks.

In another embodiment of the invention, the compound of the invention and a second active ingredient are administered orally, with administration of the compound of the invention occurring 30 to 60 minutes prior to a second active ingredient, during a cycle of four to six weeks. In another embodiment of the invention, the combination of the compound of the invention and a second active ingredient is administered by intravenous infusion over about 90 minutes every cycle. In a specific embodiment, one cycle comprises the administration of from about 0.1 to about 200 mg/day of the compound of the invention and from about 50 to about 200 mg/m²/day of a second active ingredient daily for three to four weeks and then one or two weeks of rest. In another specific embodiment, each cycle comprises the administration of from about 1 to about 25 mg/day of the compound of the invention and from about 50 to about 200 mg/m²/day of a second active ingredient for 3 to 4 weeks followed by one or two weeks of rest. Typically, the number of cycles during which the combinatorial treatment is administered to a patient will be from about one to about 24 cycles, more typically from about two to about 16 cycles and even more typically from about four to about three cycles.

The amount of the pharmaceutical composition administered according to the methods of the invention will depend on the subject being treated, the severity of the disorder or symptom of the disorder, the manner of administration, the frequency of administration and the judgment of the prescribing physician.

The frequency of administration is in the range of about an hourly dose to a monthly dose. In specific embodiments, administration is from 8 times per day to once every other day or from 1 to 3 times per day. In a specific embodiment, a pharmaceutical composition of the invention is administered chronically, e.g., daily.

It may be necessary to use dosages of the active ingredient outside the ranges disclosed herein in some cases, as will be apparent to those of ordinary skill in the art. Furthermore, it is noted that the clinician or treating physician will know how and when to interrupt, adjust, or terminate therapy in conjunction with individual patient response.

### 5.4 Doses

In one embodiment of the invention, (+)-2-[1-(3-ethoxy-4-methoxyphenyl)-2-methylsulfonylethyl]-4-acetylaminoisoindoline-1,3-dione can be administered orally and in single or divided daily doses in an amount of from about 1 mg to about 1000 mg per day, given as a single once-a-day dose, preferably as divided doses throughout a day. More specifically, the daily dose of (+)-2-[1-(3-ethoxy-4-methoxyphenyl)-2-methylsulfonylethyl]-4-acetylaminoisoindoline-1,3-dione is administered twice daily in equally divided doses. Specifically, a daily dose range of (+)-2-[1-(3-ethoxy-4-methoxyphenyl)-2-methylsulfonylethyl]-4-acetylaminoisoindoline-1,3-dione can be from about 5 mg to about 500 mg per day, more specifically, between about 10 mg and about 200 mg per day. Specifically, the daily dose of (+)-2-[1-(3-ethoxy-4-methoxyphenyl)-2-methylsulfonylethyl]-4-acetylaminoisoindoline-1,3-dione may be administered in 5 mg, 10 mg, 15 mg, 20 mg, 25 mg, 50 mg, or 100 mg dosage forms. In managing the patient, the therapy should be initiated at a lower dose, perhaps about 1 mg to about 25 mg, and increased if necessary up to about 200 mg to about 1000 mg per day as either a single dose or divided doses, depending on the patient's global response. Alternatively, the daily dose is from 0.01 mg/kg to 100 mg/kg.

In one embodiment, 4-(amino)-2-(2,6-dioxo(3-piperidyl))-isoindoline-1,3-dione can be administered in an amount of from about 0.1 to about 100 mg. In a specific embodiment, 4-(amino)-2-(2,6-dioxo(3-piperidyl))-isoindoline-1,3-dione may be administered in an amount of from about 1 to about 100 mg per day. In a particular embodiment, 4-(amino)-2-(2,6-dioxo(3-piperidyl))-isoindoline-1,3-dione may be administered in an amount of from about 0.1 to about 2 mg per day, or alternatively from about 0.1 to about 5 mg every other day. In a specific embodiment, 4-(amino)-2-(2,6-dioxo(3-piperidyl))-isoindoline-1,3-dione may be administered in an amount of from about 0.5 to about 2 mg per day, or alternatively about 5 mg every other day.

In one embodiment, 3-(4amino-1-oxo-1,3-dihydro-isoindol-2-yl)-piperidine-2,6-dione can be administered in an amount of from about 1 to about 150 mg. In a specific embodiment, 3-(4 amino-1-oxo-1,3-dihydro-isoindol-2-yl)-piperidine-2,6-dione may be administered in an amount of from about 5 to 25 mg per day, or alternatively from about 10 to about 50 mg every other day.

In further embodiment of the invention, cyclopropyl {2-[(1S)-1-(3-ethoxy-4-methoxyphenyl)-2-(methylsulfonyl)ethyl]-3-oxoisoindolin-4-yl}carboxamide can be administered orally and in single or divided daily doses in an amount of from about 1 mg to about 1000 mg per day, given as a single once-a-day dose, preferably as divided doses throughout a day. More specifically, the daily dose of cyclopropyl {2-[(1S)-1-(3-ethoxy-4-methoxyphenyl)-2-(methylsulfonyl)ethyl]-3-oxoisoindolin-4-yl}carboxamide is administered twice daily in equally divided doses. Specifically, a daily dose range of cyclopropyl {2-1-[(1S)-1-(3-ethoxy-4-methoxyphenyl)-2-(methylsulfonyl)ethyl]-3-oxoisoindolin-4-yl}carboxamide can be from about 5 mg to about 500 mg per day, more specifically, between about 10 mg and about 200 mg per day. Specifically, the daily dose of cyclopropyl {2-[(1S)-1-(3-ethoxy-4-methoxyphenyl)-2-(methylsulfonyl)ethyl]-3-oxoisoindolin-4-yl}carboxamide may be administered in 5 mg, 10 mg, 15 mg, 20 mg, 25 mg, 50 mg, or 100 mg dosage forms. In managing the patient, the therapy should be initiated at a lower dose, perhaps about 1 mg to about 25 mg, and increased if necessary up to about 200 mg to about 1000 mg per day as either a single dose or divided doses, depending on the patient's global response. Alternatively, the daily dose is from 0.01 mg/kg to 100 mg/kg.

Various dosage forms of the invention are discussed in section 5.5 below. In one embodiment, typical dosage forms of the invention comprise (+)-2-[1-(3-ethoxy-4-methoxyphenyl)-2-methylsulfonylethyl]-4-acetylaminoisoindoline-1,3-dione, or cyclopropyl {2-[(1S)-1-(3-ethoxy-4-methoxyphenyl)-2-(methylsulfonyl)ethyl]-3-oxoisoindolin-4-yl}carboxamide, in an amount from about 0.10 to about 1000 mg, from about 0.10 to about 800 mg, from about 0.10 to about 600 mg, from about 0.10 to about 500 mg, from about 0.10 to about 400 mg, from about 0.10 to about 300 mg, from about 0.10 to about 200 mg, or from about 0.10 to about 100 mg. In one embodiment, typical dosage forms comprise the compound in an amount of about 1, 2, 5, 10, 25, 50, 100, 150, 200, 250, 300, 350, 400, 450, or 500 mg.

In one embodiment, typical dosage forms of the invention comprise 4-(amino)-2-(2,6-dioxo(3-piperidyl))-isoindoline-1,3-dione or 3-(4-amino-1-oxo-1,3-dihydro-isoindol-2-yl)-piperidine-2,6-dione in an amount of from about 0.1 to about 150 mg. In a particular embodiment, a dosage form comprises 4-(amino)-2-(2,6-dioxo(3-piperidyl))-isoindoline-1,3-dione in an amount of about 0.1, 1, 2, or 5 mg. In a particular embodiment, a dosage form comprises 3-(4-amino-1-oxo-1,3-dihydro-isoindol-2-yl)-piperidine-2,6-dione in an amount of about 5, 10, 15, 25 or 50 mg.

In one embodiment, typical dosage forms comprise the second active ingredient in an amount of 1 to about 1000 mg, from about 5 to about 500 mg, from about 10 to about 350 mg or from about 50 to about 200 mg. Of course, the specific amount of the agent will depend on the specific agent used, the type of disease or disorder being treated or managed and the amount(s) of the compounds of the invention and any optional additional second active agents concurrently administered to the patient.

### 5.5 Pharmaceutical Compositions and Dosage Forms

Pharmaceutical compositions can be used in the preparation of individual, single unit dosage forms. Pharmaceutical compositions and dosage forms of the invention can comprise the compounds of the invention, or a pharmaceutically acceptable salt, solvate or stereoisomer thereof, and optionally a second active agent. Examples of the optional second active agents are disclosed herein (see, e.g., section 5.3.1). Pharmaceutical compositions and dosage forms of the invention can further comprise one or more carriers, excipients or diluents.

Single unit dosage forms of the invention are suitable for oral, mucosal (e.g., sublingual, nasal, vaginal, cystic, rectal, preputial, ocular, buccal or aural), parenteral (e.g., subcutaneous, intravenous, bolus injection, intramuscular or intraarterial), topical (e.g., eye drops or other ophthalmic preparations), transdermal or transcutaneous administration to a patient. Non-limiting examples of dosage forms include tablets; caplets; capsules, such as soft elastic gelatin capsules; cachets; troches; lozenges; dispersions; suppositories; powders; aerosols (e.g., nasal sprays or inhalers); gels; liquid dosage forms suitable for oral or mucosal administration to a patient, including suspensions (e.g., aqueous or non-aqueous liquid suspensions, oil-in-water emulsions or a water-in-oil liquid emulsions), solutions and elixirs; liquid dosage forms suitable for parenteral administration to a patient; eye drops or other ophthalmic preparations suitable for topical administration; and sterile solids (e.g., crystalline or amorphous solids) that can be reconstituted to provide liquid dosage forms suitable for parenteral administration to a patient.

The composition, shape and type of dosage forms of the invention will typically vary depending on their use. For example, a dosage form used in the acute treatment of a disease may contain larger amounts of one or more of the active ingredients it comprises than a dosage form used in the chronic treatment of the same disease. Similarly, a parenteral dosage form may contain smaller amounts of one or more of the active ingredients it comprises than an oral dosage form used to treat the same disease. These and other ways in which specific dosage forms encompassed by this invention will vary from one another will be readily apparent to those skilled in the art. See, e.g., Remington's Pharmaceutical Sciences, 18th ed., Mack Publishing, Easton Pa. (1990).

Typical pharmaceutical compositions and dosage forms comprise one or more excipients. Suitable excipients are well known to those skilled in the art of pharmacy and non-limiting examples of suitable excipients are provided herein. Whether a particular excipient is suitable for incorporation into a pharmaceutical composition or dosage form depends on a variety of factors well known in the art including, but not limited to, the way in which the dosage form will be administered to a patient. For example, oral dosage forms such as tablets may contain excipients not suited for use in parenteral dosage forms. The suitability of a particular excipient may also depend on the specific active ingredients in the dosage form. For example, the decomposition of some active ingredients can be accelerated by some excipients such as lactose or when exposed to water. Active ingredients that comprise primary or secondary amines are particularly susceptible to such accelerated decomposition. Consequently, this invention encompasses pharmaceutical compositions and dosage forms that contain little, if any, lactose or other mono- or di-saccharides. As used herein, the term “lactose-free” means that the amount of lactose present, if any, is insufficient to substantially increase the degradation rate of an active ingredient.

Lactose-free compositions of the invention can comprise excipients that are well known in the art and are listed, for example, in the U.S. Pharmacopeia (USP) 25-NF20 (2002). In general, lactose-free compositions comprise active ingredients, a binder/filler and a lubricant in pharmaceutically compatible and pharmaceutically acceptable amounts. Particular lactose-free dosage forms comprise active ingredients, microcrystalline cellulose, pre-gelatinized starch and magnesium stearate.

This invention further encompasses anhydrous pharmaceutical compositions and dosage forms comprising active ingredients, since water can facilitate the degradation of some compounds. For example, the addition of water (e.g., 5%) is widely accepted in the pharmaceutical arts as a means of simulating long-term storage in order to determine characteristics such as shelf-life or the stability of formulations over time. See, e.g., Jens T. Carstensen, Drug Stability: Principles & Practice, 2d. Ed., Marcel Dekker, NY, N.Y., 1995, pp. 379-80. In effect, water and heat accelerate the decomposition of some compounds. Thus, the effect of water on a formulation can be of great significance since moisture and/or humidity are commonly encountered during manufacture, handling, packaging, storage, shipment and use of formulations.

Anhydrous pharmaceutical compositions and dosage forms of the invention can be prepared using anhydrous or low moisture containing ingredients and low moisture or low humidity conditions. Pharmaceutical compositions and dosage forms that comprise lactose and at least one active ingredient that comprises a primary or secondary amine are preferably anhydrous if substantial contact with moisture and/or humidity during manufacturing, packaging and/or storage is expected.

An anhydrous pharmaceutical composition should be prepared and stored such that its anhydrous nature is maintained. Accordingly, anhydrous compositions are preferably packaged using materials known to prevent exposure to water such that they can be included in suitable formulary kits. Non-limiting examples of suitable packaging include hermetically sealed foils, plastics, unit dose containers (e.g., vials), blister packs and strip packs.

The invention further encompasses pharmaceutical compositions and dosage forms that comprise one or more compounds that reduce the rate by which an active ingredient will decompose. Such compounds, which are referred to herein as “stabilizers,” include, but are not limited to, antioxidants such as ascorbic acid, pH buffers or salt buffers. Like the amounts and types of excipients, the amounts and specific types of active ingredients in a dosage form may differ depending on factors such as, but not limited to, the route by which it is to be administered to patients.

5.5.1 Oral Dosage Forms

Pharmaceutical compositions of the invention that are suitable for oral administration can be presented as discrete dosage forms, such as, but are not limited to, tablets (e.g., chewable tablets), caplets, capsules and liquids (e.g., flavored syrups). Such dosage forms contain predetermined amounts of active ingredients and can be prepared by methods of pharmacy well known to those skilled in the art. See generally, Remington's Pharmaceutical Sciences, 18th ed., Mack Publishing, Easton Pa. (1990).

Typical oral dosage forms of the invention are prepared by combining the active ingredients in an intimate admixture with at least one excipient according to conventional pharmaceutical compounding techniques. Excipients can take a wide variety of forms depending on the form of preparation desired for administration. Non-limiting examples of excipients suitable for use in oral liquid or aerosol dosage forms include water, glycols, oils, alcohols, flavoring agents, preservatives and coloring agents. Non-limiting examples of excipients suitable for use in solid oral dosage forms (e.g., powders, tablets, capsules and caplets) include starches, sugars, micro-crystalline cellulose, diluents, granulating agents, lubricants, binders and disintegrating agents.

Because of their ease of administration, tablets and capsules represent the most advantageous oral dosage unit forms, in which case solid excipients are employed. If desired, tablets can be coated by standard aqueous or nonaqueous techniques. Such dosage forms can be prepared by any of the methods of pharmacy. In general, pharmaceutical compositions and dosage forms are prepared by uniformly and intimately admixing the active ingredients with liquid carriers, finely divided solid carriers or both and then shaping the product into the desired presentation if necessary.

For example, a tablet can be prepared by compression or molding. Compressed tablets can be prepared by compressing in a suitable machine the active ingredients in a free-flowing form such as powder or granules, optionally mixed with an excipient. Molded tablets can be made by molding in a suitable machine a mixture of the powdered compound moistened with an inert liquid diluent.

Non-limiting examples of excipients that can be used in oral dosage forms of the invention include binders, fillers, disintegrants and lubricants. Non-limiting examples of binders suitable for use in pharmaceutical compositions and dosage forms include corn starch, potato starch or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, (e.g., Nos. 2208, 2906, 2910), microcrystalline cellulose and mixtures thereof.

Non-limiting examples of suitable forms of microcrystalline cellulose include the materials sold as AVICEL-PH-101, AVICEL-PH-103 AVICEL RC-581, AVICEL-PH-105 (available from FMC Corporation, American Viscose Division, Avicel Sales, Marcus Hook, Pa.) and mixtures thereof. An specific binder is a mixture of microcrystalline cellulose and sodium carboxymethyl cellulose sold as AVICEL RC-581. Suitable anhydrous or low moisture excipients or additives include AVICEL-PH-103™ and Starch 1500 LM.

Non-limiting examples of fillers suitable for use in the pharmaceutical compositions and dosage forms disclosed herein include talc, calcium carbonate (e.g., granules or powder), microcrystalline cellulose, powdered cellulose, dextrates, kaolin, mannitol, silicic acid, sorbitol, starch, pre-gelatinized starch and mixtures thereof. The binder or filler in pharmaceutical compositions of the invention is typically present in from about 50 to about 99 weight percent of the pharmaceutical composition or dosage form.

Disintegrants are used in the compositions of the invention to provide tablets that disintegrate when exposed to an aqueous environment. Tablets that contain too much disintegrant may disintegrate in storage, while those that contain too little may not disintegrate at a desired rate or under the desired conditions. Thus, a sufficient amount of disintegrant that is neither too much nor too little to detrimentally alter the release of the active ingredients should be used to form solid oral dosage forms of the invention. The amount of disintegrant used varies based upon the type of formulation and is readily discernible to those of ordinary skill in the art. Typical pharmaceutical compositions comprise from about 0.5 to about 15 weight percent of disintegrant, preferably from about 1 to about 5 weight percent of disintegrant.

Non-limiting examples of disintegrants that can be used in pharmaceutical compositions and dosage forms of the invention include agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums and mixtures thereof.

Non-limiting examples of lubricants that can be used in pharmaceutical compositions and dosage forms of the invention include calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil and soybean oil), zinc stearate, ethyl oleate, ethyl laureate, agar and mixtures thereof. Additional lubricants include, for example, a syloid silica gel (AEROSIL200, manufactured by W.R. Grace Co. of Baltimore, Md.), a coagulated aerosol of synthetic silica (marketed by Degussa Co. of Plano, Tex.), CAB-O-SIL (a pyrogenic silicon dioxide product sold by Cabot Co. of Boston, Mass.) and mixtures thereof. If used at all, lubricants are typically used in an amount of less than about 1 weight percent of the pharmaceutical compositions or dosage forms into which they are incorporated.

A particular solid oral dosage form of the invention comprises the compound of the invention (e.g., (+)-2-[1-(3-ethoxy-4-methoxyphenyl)-2-methylsulfonylethyl]-4-acetylaminoisoindoline-1,3-dione, 4-(amino)-2-(2,6-dioxo(3-piperidyl))-isoindoline-1,3-dione, 3-(4-amino-1-oxo-1,3-dihydro-isoindol-2-yl)-piperidine-2,6-dione, or cyclopropyl {2-[(1S)-1-(3-ethoxy-4-methoxyphenyl)-2-(methylsulfonyl)ethyl]-3-oxoisoindolin-4-yl}carboxamide), anhydrous lactose, microcrystalline cellulose, polyvinylpyrrolidone, stearic acid, colloidal anhydrous silica and gelatin.

5.5.2 Delayed Release Dosage Forms

Active ingredients of the invention can be administered by controlled release means or by delivery devices that are well known to those of ordinary skill in the art. Non-limiting examples of controlled release means or delivery devices include those described in U.S. Pat. Nos.: 3,845,770; 3,916,899; 3,536,809; 3,598,123; and 4,008,719, 5,674,533, 5,059,595, 5,591,767, 5,120,548, 5,073,543, 5,639,476, 5,354,556 and 5,733,566, each of which is incorporated herein by reference. Such dosage forms can be used to provide slow or controlled-release of one or more active ingredients using, for example, hydropropylmethyl cellulose, other polymer matrices, gels, permeable membranes, osmotic systems, multilayer coatings, microparticles, liposomes, microspheres or a combination thereof to provide the desired release profile in varying proportions. Suitable controlled-release formulations known to those of ordinary skill in the art, including those described herein, can be readily selected for use with the active ingredients of the invention. The invention thus encompasses single unit dosage forms suitable for oral administration such as, but not limited to, tablets, capsules, gelcaps and caplets that are adapted for controlled-release.

All controlled-release pharmaceutical products have a common goal of improving drug therapy over that achieved by their non-controlled counterparts. Ideally, the use of an optimally designed controlled-release preparation in medical treatment is characterized by a minimum of drug substance being employed to cure or control the condition in a minimum amount of time. Advantages of controlled-release formulations include extended activity of the drug, reduced dosage frequency and increased patient compliance. In addition, controlled-release formulations can be used to affect the time of onset of action or other characteristics, such as blood levels of the drug and can thus affect the occurrence of side (e.g., adverse) effects.

Most controlled-release formulations are designed to initially release an amount of drug (active ingredient) that promptly produces the desired therapeutic effect and gradually and continually release of other amounts of drug to maintain this level of therapeutic or prophylactic effect over an extended period of time. In order to maintain this constant level of drug in the body, the drug must be released from the dosage form at a rate that will replace the amount of drug being metabolized and excreted from the body. Controlled-release of an active ingredient can be stimulated by various conditions including, but not limited to, pH, temperature, enzymes, water or other physiological conditions or compounds.

5.5.3 Parenteral Dosage Forms

Parenteral dosage forms can be administered to patients by various routes including, but not limited to, subcutaneous, intravenous (including bolus injection), intramuscular and intraarterial. Because their administration typically bypasses patients' natural defenses against contaminants, parenteral dosage forms are preferably sterile or capable of being sterilized prior to administration to a patient. Non-limiting examples of parenteral dosage forms include solutions ready for injection, dry products ready to be dissolved or suspended in a pharmaceutically acceptable vehicle for injection, suspensions ready for injection and emulsions.

Suitable vehicles that can be used to provide parenteral dosage forms of the invention are well known to those skilled in the art. Non-limiting examples of suitable vehicles include Water for Injection USP; aqueous vehicles such as, but not limited to, Sodium Chloride Injection, Ringer's Injection, Dextrose Injection, Dextrose and Sodium Chloride Injection and Lactated Ringer's Injection; water-miscible vehicles such as, but not limited to, ethyl alcohol, polyethylene glycol and polypropylene glycol; and non-aqueous vehicles such as, but not limited to, corn oil, cottonseed oil, peanut oil, sesame oil, ethyl oleate, isopropyl myristate and benzyl benzoate.

Compounds that increase the solubility of one or more of the active ingredients disclosed herein can also be incorporated into the parenteral dosage forms of the invention. For example, cyclodextrin and its derivatives can be used to increase the solubility of the compounds of the invention and its derivatives.

5.5.4 Topical, Transdermal and Mucosal Dosage Forms

Drugs can be applied locally to the skin and its adnexa or to a variety of mucous membranes. The routes that can be used include topical, transdermal, sublingual, nasal, vaginal, cystic, rectal, preputial, ocular, buccal or aural. Many dosage forms have been developed to deliver active principles to the site of application to produce local effects. Transdermal, topical, and mucosal dosage forms of the invention include, but are not limited to, ophthalmic solutions, sprays, aerosols, creams, lotions, ointments, gels, solutions, emulsions, suspensions, or other forms known to one of skill in the art. See, e.g., Remington's Pharmaceutical Sciences, 16th and 18th eds., Mack Publishing, Easton Pa. (1980 & 1990); and Introduction to Pharmaceutical Dosage Forms, 4th ed., Lea & Febiger, Philadelphia (1985). Dosage forms suitable for treating mucosal tissues within the oral cavity can be formulated as mouthwashes or as oral gels. Further, transdermal dosage forms include “reservoir type” or “matrix type” patches, which can be applied to the skin and worn for a specific period of time to permit the penetration of a desired amount of active ingredients.

Suitable excipients (e.g., carriers and diluents) and other materials that can be used to provide transdermal, topical, and mucosal dosage forms encompassed by this invention are well known to those skilled in the pharmaceutical arts, and depend on the particular tissue to which a given pharmaceutical composition or dosage form will be applied. With that fact in mind, typical excipients include, but are not limited to, water, acetone, ethanol, ethylene glycol, propylene glycol, butane 1,3diol, isopropyl myristate, isopropyl palmitate, mineral oil, and mixtures thereof to form lotions, tinctures, creams, emulsions, gels or ointments, which are non toxic and pharmaceutically acceptable. Moisturizers such as occlusives, humectants, emollients and protein rejuvenators can also be added to pharmaceutical compositions and dosage forms if desired. Examples of such additional ingredients are well known in the art. See, e.g., Remington's Pharmaceutical Sciences, 16th and 18th eds., Mack Publishing, Easton Pa. (1980 & 1990).

Occlusives are substances that physically block water loss in the stratum corneum. Non-limiting examples of occlusives include petrolatum, lanolin, mineral oil, silicones such as dimethicone, zinc oxide and combinations thereof. Preferably, the occlusives are petrolatum and lanolin, more preferably petrolatum in a minimum concentration of 5%.

Humectants are substances that attract water when applied to the skin and theoretically improve hydration of the stratum corneum. However, the water that is drawn to the skin is water from other cells, not atmospheric water. With this type of moisturizer, evaporation from the skin can continue and actually can make the dryness worse. Non-limiting examples of humectants include glycerin, sorbitol, urea, alpha hydroxy acids, sugars and combinations thereof. Preferably, the humectants are alpha hydroxy acids, such as glycolic acid, lactic acid, malic acid, citric acid and tartaric acid.

Emollients are substances that smooth skin by filling spaces between skin flakes with droplets of oil, and are not usually occlusive unless applied heavily. When combined with an emulsifier, they may help hold oil and water in the stratum corneum. Vitamin E is a common additive, which appears to have no effect, except as an emollient. Likewise, other vitamins, for example, A and D, are also added, but their effect is questionable. Non-limiting examples of emollients include mineral oil, lanolin, fatty acids, cholesterol, squalene, structural lipids and combinations thereof.

Protein rejuvenators are substances that rejuvenate the skin by replenishing essential proteins. Non-limiting examples of protein rejuvenators include collagen, keratin, elastin and combinations thereof.

Depending on the specific tissue to be treated, additional components may be used prior to, in conjunction with, or subsequent to treatment with active ingredients of the invention. For example, penetration enhancers can be used to assist in delivering the active ingredients to the tissue. Suitable penetration enhancers include, but are not limited to: acetone; various alcohols such as ethanol, oleyl, and tetrahydrofuryl; alkyl sulfoxides such as dimethyl sulfoxide; dimethyl acetamide; dimethyl formamide; polyethylene glycol; pyrrolidones such as polyvinylpyrrolidone; Kollidon grades (Povidone, Polyvidone); urea; and various water soluble or insoluble sugar esters such as Tween 80 (polysorbate 80) and Span 60 (sorbitan monostearate).

The pH of a pharmaceutical composition or dosage form may also be adjusted to improve delivery of one or more active ingredients. Similarly, the polarity of a solvent carrier, its ionic strength or tonicity can be adjusted to improve delivery. For example, absorption through the skin can also be enhanced by occlusive dressings, inunction or the use of dimethyl sulfoxide as a carrier. Compounds such as metal stearates (e.g., calcium stearate, zinc stearate, magnesium stearate, sodium stearate, lithium stearate, potassium stearate, etc.) can also be added to pharmaceutical compositions or dosage forms to advantageously alter the hydrophilicity or lipophilicity of one or more active ingredients so as to improve delivery. In this regard, stearates can serve as a lipid vehicle for the formulation, as an emulsifying agent or surfactant and as a delivery-enhancing or penetration-enhancing agent. Different salts, hydrates or solvates of the active ingredients can be used to further adjust the properties of the resulting composition.

### 6. EXAMPLES

Some embodiments of the invention are illustrated by the following non-limiting examples. The examples should not be construed as a limitation in the scope thereof. The scope of the invention is defined solely by the appended claims.

### Example 1

### Preparation of (+)-2-[1-(3-ethoxy-4-methoxyphenyl)-2-methylsulfonylethyl]-4-acetylaminoisoindoline-1,3-dione [Compound (1)]

Preparation of 3-Aminophthalic acid. After a mixture of 10% Pd/C (2.5 g), 3-nitrophthalic acid (75.0 g, 355 mmol) and ethanol (1.5 L) was charged to a 2.5 L Parr hydrogenator under nitrogen, hydrogen was charged to the reaction vessel for up to 55 psi (379 kPa). The mixture was shaken for 13 hours while the hydrogen pressure was maintained at between 50 psi (245 kPa) and 55 psi (379 kPa). Hydrogen was released and the mixture was purged with nitrogen 3 times. The suspension was filtered through a celite bed and rinsed with methanol. The filtrate was concentrated in vacuum to yield a solid. The solid was suspended in ether and isolated by vacuum filtration. The solid was dried in vacuum to a constant weight to afford 54 g (84% yield) of 3-aminopthalic acid as a yellow product. The product in DMSO-d₆ was characterized by a ¹H NMR spectrum showing the following chemical shifts (δ in ppm): 3.17 (s, 2H), 6.67 (d, 1H), 6.82 (d, 1H), 7.17 (t, 1H), 8-10 (brs, 2H). The product in DMSO-d₆ was characterized by a ¹³C-NMR spectrum showing the following chemical shifts (δ in ppm): 112.00, 115.32, 118.20, 131.28, 135.86, 148.82, 169.15, 170.09.

Preparation of 3-acetamidophthalic anhydride. A mixture of 3-aminophthalic acid (108 g, 596 mmol) and acetic anhydride (550 mL) was charged into a 1-L 3-necked round bottom flask equipped with a mechanical stirrer, a thermometer, and a condenser. The reaction mixture was refluxed for 3 hours, cooled to ambient temperature, and kept at 0-5° C. for another 1 hour. The crystalline solid was collected by vacuum filtration and washed with ether. The solid product was dried in vacuum at ambient temperature to a constant weight to yield 75 g (61% yield) of 3-acetamidopthalic anhydride as a white product. The product in CDCl₃ was characterized by a ¹H NMR spectrum showing the following chemical shifts (δ in ppm): 2.21 (s, 3H), 7.76 (d, 1H), 7.94 (t, 1H), 8.42 (d, 1H), 9.84 (s, 1H).

Resolution of 2-(3-ethoxy-4-methoxyphenyl)-1-(methylsulphonyl)-eth-2-ylamine. A mixture of 2-(3-ethoxy-4-methoxyphenyl)-1-(methylsulphonyl)-eth-2-ylamine (137.0 g, 500 mmol), N-acetyl-L-leucine (52 g, 300 mmol), and methanol (1.0 L) was charged into a 3-L 3-necked round bottom flask equipped with a mechanical stirrer, a thermometer, and a condenser. After the reaction mixture was refluxed for 1 hour, the mixture was allowed to cool to ambient temperature and then stirred for another 3 hours at ambient temperature. The slurry was filtered and washed with methanol (250 L). The solid was air-dried and then dried in vacuum at ambient temperature to a constant weight, giving 109.5 g (98% yield) of the crude product (85.8% ee). The crude solid (55.0 g) and methanol (440 mL) were brought to reflux for 1 hour, cooled to room temperature and stirred for an additional 3 hours at ambient temperature. The slurry was filtered and the filter cake was washed with methanol (200 mL). The solid was air-dried and then dried in vacuum at 30° C. to a constant weight, yielding 49.6 g (90% recovery) of (S)-2-(3-ethoxy-4-methoxyphenyl)-1-(methylsulphonyl)-eth-2-ylamine-N-acetyl-L-leucine salt (98.4% ee). Chiral HPLC (1/99 EtOH/20 mM KH2PO4 @pH 7.0, Ultron Chiral ES-OVS from Agilent Technologies, 150 mm×4.6 mm, 0.5 mL/min., @240 nm): 18.4 min (S-isomer, 99.2%), 25.5 min (R-isomer, 0.8%).

Preparation of (+)-2-[1-(3-Ethoxy-4-methoxyphenyl)-2-methylsulfonylethyl]-4-acetylaminoisoindoline-1,3-dione. A 500 mL 3-necked round bottom flask was equipped with a mechanical stirrer, thermometer, and condenser. The reaction vessel was charged with (S)-2-(3-ethoxy-4-methoxyphenyl)-1-(methylsulphonyl)-eth-2-yl amine N-acetyl-L-leucine salt (25 g, 56 mmol, 98% ee), 3-acetamidophthalic anhydride (12.1 g 58.8 mmol), and glacial acetic acid (250 mL). The mixture was refluxed over night and then cooled to <50° C. After the solvent was removed in vacuum, the residue was dissolved in ethyl acetate. The resulting solution was washed with water (250 mL×2), saturated aqeous NaHCO₃ (250 mL×2), and brine (250 mL×2), and then dried over anhydrous sodium sulfate. After the solvent was evaporated in vacuum, the residue was recrystallized from a binary solvent containing a mixture of ethanol (150 mL) and acetone (75 mL). The solid was isolated by vacuum filtration and washed with ethanol (100 mL×2). The product was dried in vacuum at 60° C. to a constant weight, affording 19.4 g (75% yield) of (S)-{2-[1-(3-ethoxy-4-methoxyphenyl)-2-methylsulfonylethyl]-4-inoisoindoline-1,3-dione with 98% ee. Chiral HPLC (15/85 EtOH/20 mM KH₂PO₄ @pH 0.5, Ultron Chiral ES-OVS from Agilent Technology, 150 mm×4.6 mm, 0.4 mL/min., @240 nm): 25.4 min (S-isomer, 98.7%), 29.5 min (R-isomer, 1.2%). The product in CDCl₃ was characterized by a ¹H NMR spectrum showing the following chemical shifts (δ in ppm): 1.47 (t, 3H), 2.26 (s, 3H), 2.87 (s, 3H), 3.68-3.75 (dd, 1H), 3.85 (s, 3H), 4.07-4.15 (q, 2H), 4.51-4.61 (dd, 1H), 5.84-5.90 (dd, 1H), 6.82-8.77 (m, 6H), 9.46 (s, 1H). The product in DMSO-d₆ was characterized by a ¹³C NMR spectrum showing the following chemical shifts (δ in ppm): 14.66, 24.92, 41.61, 48.53, 54.46, 55.91, 64.51, 111.44, 112.40, 115.10, 118.20, 120.28, 124.94, 129.22, 131.02, 136.09, 137.60, 148.62, 149.74, 167.46, 169.14, 169.48.

### Example 2

### Preparation of 4-amino-2-(2,6-dioxo-3-piperidinyl) isoindole-1,3-dione [Compound (2)]

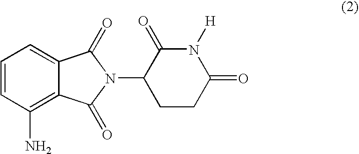

To a round bottom flask equipped with a mechanical stirrer, a condenser, a nitrogen inlet and a heating mantel was charged with a mixture of acetonitrile (42 L) and N-(3-aminophthaloyl)-glutamine (2120 g, 7.28 moles). After the mixture was stirred and heated to 40-45° C., 1,1′-carbonyldiimidazole (1290 g, 7.95 moles) was added. The reaction mixture was stirred and refluxed for 4.5 hours. The progress of the reaction was monitored by HPLC using a Waters Nova-Pak C18 column (3.9×150 mm, particle size=4 micron, UV wavelength=240 nm, retention time=3.64 minutes) and a 20/80 mixture of acetonitrile and 0.1% aqueous H₃PO₄ by volume as an eluent at a flow rate of 1 mL/min. After cooled to room temperature, the reaction mixture was filtered to yield a yellow solid which was subsequently washed with acetonitrile (6.5 L). The yellow solid was air dried and then dried in a vacuum oven at 60° C. and a pressure <1 mm to yield 1760 g (88%) of the product. The product purity was found to be 99.57% by HPLC using a Waters Nova-Pak C18 column (3.9×150 mm, particle size=4 micron, UV wavelength=240 nm, retention time=3.64 minutes) and a 20/80 mixture of acetonitrile and 0.1% aqueous H₃PO₄ by volume as an eluent at a flow rate of 1 mL/min. The product in DMSO-d₆ was characterized by a ¹H NMR spectrum showing the following chemical shifts (δ in ppm): 11.10 (s, 1H), 7.47(t, J=7.9 Hz, 1H), 7.03-6.99 (dd, J=4.8 and 8.4 Hz, 2H), 6.52 (s, 2H), 5.09-5.02 (dd, J=5.3 and 12.4 Hz, 1H), 2.96-2.82 (m, 1H), 2.62-2.46 (m, 2H), 2.07-2.00 (m, 1H); and by a ¹³C NMR spectrum showing the following chemical shifts (δ in ppm): 172.82, 170.11, 168.57, 167-37, 146.71, 135.46, 131.99, 121.70, 110.97, 108.52, 48.47, 30.97, 22.14. The melting point of the product was found to be 315.5-317.5° C. An elemental analysis yielded the following results in weight percent: C, 56.98; H, 3.86; N, 15.35, which compared with calculated values for C₁₃H₁₁N₃O₄, in weight percent: 57.14; H, 4.06; N, 15.38.

### Example 3

### Preparation of cyclopropyl {2-[(1S)-1-(3-ethoxy-4-methoxyphenyl)-2-(methylsulfonyl)ethyl]-3-oxoisoindolin-4yl}carboximide

Cyclopropyl {2-[(1S)-1-(3-ethoxy-4-methoxyphenyl)-2-(methylsulfonyl)ethyl]-3-oxoisoindolin-4-yl}carboxamide was prepared according to the preparation procedure for Example 57 of U.S. Pat. No. 6,667,316. A stirred mixture of 7-amino-2-[(1S)-1-(3-ethoxy-4-methoxyphenyl)-2-(methylsulfonyl)ethyl]isoindolin-1-one (1.7 g, 4.2 mmol) and cyclopropanecarbonyl chloride (0.46 mL, 5.1 mmol) in tetrahydrofuran (10 mL) was heated to reflux for 15 minutes. To the mixture was added methanol (4 mL) at room temperature and the mixture was stirred for 10 minutes. The solvent was removed in vacuo to yield an oil. The oil was recrystallized from ethanol (20 mL) to give Compound (1) as a white solid (1.4 g, 71% yield); m.p. 172-174° C.; ¹H NMR (CDCl₃) δ: 0.86-0.93 (m, 2H, 2CHH), 1.07-1.14 (m, 2H, 2CHH), 1.46 (t, J=6.9 Hz, 3H, CH₃), 1.63-1.73 (m, 1H, CH), 2.95 (s, 3H, CH₃), 3.68 (dd, J=4.4, 14.3 Hz, 1H, CCH), 3.86 (s, 3H, CH₃), 4.07 (q, J=7.1 Hz, 2H, CH₂), 4.20 (d, J=16.7 Hz, 1H, CHH), 4.21 (dd, J=9.9, 14.3 Hz, 1H, CHH), 4.44 (d, J=16.7 Hz, 1H, CHH), 5.73 (dd, J=4.3, 9.9 Hz, 1H, NCH), 6.84-7.02 (m, 4H, Ar), 7.44 (t, J=7.8 Hz, 1H, Ar), 8.43 (d, J=8.3 Hz, 1H, Ar), 10.46 (s, 1H, NH); ¹³C NMR (CDCl₃) δ: 8.24, 14.61, 16.10, 41.43, 47.81, 51.55, 55.75, 55.88, 64.56, 111.46, 112.09, 116.69, 116.99, 117.76, 119.17, 129.27, 133.54, 138.06, 141.22, 148.84, 149.67, 169.96, 172.59; Anal. Calcd. for C₂₄H₂₈N₂O₆S: C, 61.00; H, 5.97; N, 5.93. Found: C, 60.87; H, 6.13; N, 6.12.

### Example 4

Tablets, each containing 50 milligrams of active ingredient, can be prepared in the following manner:

Composition (for 1000 tablets)

| | |
|---|---|
| active ingredient | 50.0 grams |
| lactose | 50.7 grams |
| wheat starch | 7.5 grams |
| polyethylene glycol 6000 | 5.0 grams |
| talc | 5.0 grams |
| magnesium stearate | 1.8 grams |
| demineralized water | q.s. |

The solid ingredients are first forced through a sieve of 0.6 mm mesh width. The active ingredient, the lactose, the talc, the magnesium stearate and half of the starch then are mixed. The active ingredient is the compound of the invention, or a pharmaceutically acceptable salt, solvate or stereoisomer thereof. The other half of the starch is suspended in 40 milliliters of water and this suspension is added to a boiling solution of the polyethylene glycol in 100 milliliters of water. The resulting paste is added to the pulverulent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### Example 5

Tablets, each containing 100 milligrams of active ingredient, can be prepared in the following manner:

Composition (for 1000 tablets)

| | |
|---|---|
| active ingredient | 100.0 grams |
| lactose | 100.0 grams |
| wheat starch | 47.0 grams |
| magnesium stearate | 3.0 grams |

All the solid ingredients are first forced through a sieve of 0.6 mm mesh width. The active ingredient, the lactose, the magnesium stearate and half of the starch then are mixed. The other half of the starch is suspended in 40 milliliters of water and this suspension is added to 100 milliliters of boiling water. The resulting paste is added to the pulverulent substances and the mixture is granulated, if necessary with the addition of water. The granulate is dried overnight at 35° C., forced through a sieve of 1.2 mm mesh width and compressed to form tablets of approximately 6 mm diameter which are concave on both sides.

### Example 6

Tablets for chewing, each containing 75 milligrams of active ingredient, can be prepared in the following manner:

Composition (for 1000 tablets)

| | |
|---|---|
| active ingredient | 75.0 grams |
| mannitol | 230.0 grams |
| lactose | 150.0 grams |
| talc | 21.0 grams |
| glycine | 12.5 grams |
| stearic acid | 10.0 grams |
| saccharin | 1.5 grams |
| 5% gelatin solution | q.s. |

All the solid ingredients are first forced through a sieve of 0.25 mm mesh width. The mannitol and the lactose are mixed, granulated with the addition of gelatin solution, forced through a sieve of 2 mm mesh width, dried at 50° C. and again forced through a sieve of 1.7 mm mesh width. The active ingredient, the glycine and the saccharin are carefully mixed. The mannitol, the lactose granulate, the stearic acid and the talc are added and the whole is mixed thoroughly and compressed to form tablets of approximately 10 mm diameter which are concave on both sides and have a breaking groove on the upper side.

### Example 7

Tablets, each containing 10 milligrams of active ingredient, can be prepared in the following manner:

Composition (for 1000 tablets)

| | |
|---|---|
| active ingredient | 10.0 grams |
| lactose | 328.5 grams |
| corn starch | 17.5 grams |
| polyethylene glycol 6000 | 5.0 grams |
| talc | 25.0 grams |
| magnesium stearate | 4.0 grams |
| demineralized water | q.s. |

The solid ingredients are first forced through a sieve of 0.6 mm mesh width. Then the active ingredient, lactose, talc, magnesium stearate and half of the starch are intimately mixed. The other half of the starch is suspended in 65 milliliters of water and this suspension is added to a boiling solution of the polyethylene glycol in 260 milliliters of water. The resulting paste is added to the pulverulent substances, and the whole is mixed and granulated, if necessary with the addition of water. The granulate is dried overnight at 35° C., forced through a sieve of 1.2 mm mesh width and compressed to form tablets of approximately 10 mm diameter which are concave on both sides and have a breaking notch on the upper side.

### Example 8

Gelatin dry-filled capsules, each containing 100 milligrams of active ingredient, can be prepared in the following manner:

Composition (for 1000 capsules)

| | |
|---|---|
| active ingredient | 100.0 grams |
| microcrystalline cellulose | 30.0 grams |
| sodium lauryl sulphate | 2.0 grams |
| magnesium stearate | 8.0 grams |

The sodium lauryl sulphate is sieved into the active ingredient through a sieve of 0.2 mm mesh width and the two components are intimately mixed for 10 minutes. The microcrystalline cellulose is then added through a sieve of 0.9 mm mesh width and the whole is again intimately mixed for 10 minutes. Finally, the magnesium stearate is added through a sieve of 0.8 mm width and, after mixing for a further 3 minutes, the mixture is introduced in portions of 140 milligrams each into size 0 (elongated) gelatin dry-fill capsules.

### Example 9

A 0.2% injection or infusion solution can be prepared, for example, in the following manner:

Composition

| | | |
|---|---|---|
| active ingredient | 5.0 | grams |
| sodium chloride | 22.5 | grams |
| phosphate buffer pH 7.4 | 300.00 | grams |
| demineralized water | to 2500.0 | milliliters |

The active ingredient is dissolved in 1000 milliliters of water and filtered through a microfilter. The buffer solution is added and the whole is made up to 2500 milliliters with water. To prepare dosage unit forms, portions of 1.0 or 2.5 milliliters each are introduced into glass ampoules (each containing respectively 2.0 or 5.0 milligrams of active ingredient).

### Example 10

An ointment for topical use can be prepared, for example, in the following manner:

Composition

| | | |
|---|---|---|
| active ingredient | 10 | g |
| petrolatum | 80 | g |
| mineral oil | 120 | g |
| 2% saline solution | 2 | L |
| triamcinolone acetonide | 0.5 | g |

The above ingredients are mixed uniformly to form an ointment using a conventional mixer or homogenizer, by shaking or by ultrasonic energy.

### Example 11

A gel for topical use can be prepared, for example, in the following manner:

Composition

| | | |
|---|---|---|
| active ingredient | 10 | g |
| Carboxylmethyl cellulose | 0.2 | g |
| Glycerin | 40.0 | g |
| 0.4 mole/L Citrate buffer | 25.0 | g |
| Distilled water | to 100 | g |

The above ingredients are mixed uniformly to form a gel using a conventional mixer or homogenizer, by shaking or by ultrasonic energy.

### Example 12

A paste for topical use can be prepared, for example, in the following manner:

Composition

| | | |
|---|---|---|
| active ingredient | 10 | g |
| Carboxymethyl cellulose | 2.0 | g |
| Glycerin | 25.0 | g |
| Cetanol | 2.8 | g |
| Glyceryl monostearate | 9.3 | g |
| Tween 80 | 2.0 | g |
| Glucuronic acid | 1.0 | g |
| 0.4 mole/l Citrate buffer | 20.0 | g |
| Distilled water | to 100 | g |

The above ingredients are mixed uniformly to form a paste using a conventional mixer or homogenizer, by shaking or by ultrasonic energy.

### Example 13

A liquid composition for topical use can be prepared, for example, in the following manner:

Composition

| | | |
|---|---|---|
| active ingredient | 10 | g |
| Carboxymethyl cellulose | 0.1 | g |
| Glycerin | 15.0 | g |
| 0.4 mole/l Citrate buffer (pH 4.5) | 50.0 | g |
| Distilled water | to 100 | g |

The solid ingredients are dispersed/dissolved in the liquid ingredients uniformly to form a liquid using a conventional mixer or homogenizer, by shaking or by ultrasonic energy.

### Example 14

A spray for topical use can be prepared, for example, in the following manner:

Composition

| | |
|---|---|
| The liquid composition of Example 12 | 100.0 g |
| Freon 114 | 100.0 g |

The liquid composition and Freon 114 are filled into Teflon-coated aluminum spray containers.

### Example 15

### Testing with Human Umbilical Vein Endothelial Cells

A) Materials. Human Umbilical Vein Endothelial Cells (HUVEC) sent from LifeBank, were tested in Experiments A-K with several adhesion molecules. The adhesion molecules tested were CD51/CD61 FITC (obtained from BD PharMingen, San Diego, Calif.; Catalog No. 555505), ICAM-1 PE also known as CD54 (obtained from BD PharMingen, San Diego, Calif.; Catalog No. 555511), ICAM-2 also known as CD102 (obtained from Research Diagnostics Inc., Concord, Mass.; Catalog No. RDI-CBL539FT), VCAM-1 (obtained from BD PharMingen, San Diego, Calif.; Catalog No. 555647), P-Selectin FITC (obtained from R&D Systems, Inc., Minneapolis, Minn.; Catalog No. BBA34), E-Selectin FITC (obtained from R&D Systems, Inc., Minneapolis, Minn.; Catalog No. BBA21), HLA Class I FITC (obtained from BD PharMingen, San Diego, Calif.; Catalog No. 555553), HLA Class II PE (obtained from BD PharMingen, San Diego, Calif.; Catalog No. 555558), CD44 FITC (obtained from BD PharMingen, San Diego, Calif.; Catalog No. 347943), CD144 (Cadherin VE) (obtained from CHEMICON International, Inc., Temecula, Calif.; Catalog No. MAB1989), IgG2a FITC (obtained from BD PharMingen, San Diego, Calif.; Cat # 556652), Ms IgG2a (obtained from CHEMICON International, Inc., Temecula, Calif.; Cat. No. PP102), IgG1 FITC (obtained from BD PharMingen, San Diego, Calif.; Cat. No. 349041) and IgG1 PE (obtained from BD PharMingen, San Diego, Calif.; Cat. No. 349043).

Compound (1) ((+)-2-[1-(3-ethoxy-4-methoxyphenyl)-2-methylsulfonylethyl]-4-acetylaminoisoindoline-1,3-dione) and Compound (2) (4-(amino)-2-(2,6-dioxo(3-piperidyl))-isoindoline-1,3-dione) were obtained according to the preparation procedures disclosed herein. 16,16-Dimethyl-PGE2 (hereinafter PGE2) was obtained from BIOMOL International, L.P. (Plymouth Meeting, Pa.; Catalog No. PG-021). TNF-α was obtained from Pierce Biotechnology (Rockford, Ill.; Catalog No. RTNFA10).

B) Methods. HUVEC's were plated on 6-well plates at a concentration of 1×10⁵ cells/well in 3 ml EBM® endothelial basal media (obtained from Cambrex Corporation, East Rutherford, N.J.; Catalog No. CC-3121) and singlequots (obtained from Cambrex Corporation, East Rutherford, N.J.; Catalog No. CC-4133). The cells were incubated overnight in a 37° C. and 5% CO₂ humidified incubator to allow cells to attach. The old media was removed in next day and replaced with 3 ml fresh EBM® endothelial basal media. Then, samples of 3 μl of 10 mM of Compound (1), Compound (2), PGE2 and a mixture of Compound (1) and PGE2 were added separately to each well of the plates in duplicate to give a final concentration of 10 μM. An unstimulated DMSO control and a TNF-α-stimulated control were also added in duplicate. The plates were incubated in a 37° C. and 5% CO₂ humidified incubator for 1 hr. TNF-α (1 μg/ml) was added to each well except the DMSO control well in a volume of 3 μl to give a final concentration of 1 μg/ml. The plates were incubated overnight in a 37° C. and 5% CO₂ humidified incubator. The cells were also tested without TNF-α. The media was removed in the next day and each well was washed with 3 ml of phosphate buffered saline (PBS). Then, 3 ml of PBS containing 1 mM of EDTA (ethylenediaminetetraacetate) was added to each well to allow the cells to detach. Once the cells detached, they were gently scraped and placed in 4.5 ml Falcon tubes. The tubes were then centrifuged at 1200 RPM for 8 minutes at 4° C. The supernatant was carefully removed. Next, 50 μl of PBS-FACS buffer (5% fetal bovine serum (FBS), 0.02% sodium azide in PBS) and 20 μl of antibodies were added to all tubes as follows:

| | | | | | |
|---|---|---|---|---|---|
| Unstimulated | Stimulated | | | | PGE2 + Compound |
| (DMSO) | (TNF) | Compound (1) + TNF | PGE2 + TNF | Compound (2) + TNF | (1) + TNF |

Experiments A and B

| | | | | | |
|---|---|---|---|---|---|
| ICAM-1 PE | CD51/61 FITC | CD51/61 FITC | CD51/61 | CD51/61 FITC | CD51/61 FITC |
| | | | FITC | | |
| CD51/61 FITC | ICAM-1 PE | ICAM-1 PE | ICAM-1 PE | ICAM-1 PE | ICAM-1 PE |

Experiments C, D and E

| | | | | | |
|---|---|---|---|---|---|
| IgG1 FITC | E-Selectin | E-Selectin | E-Selectin FITC | E-Selectin FITC | E-Selectin FITC |
| | FITC | FITC | | | |
| E-Selectin FITC | P-Selectin | P-Selectin | P-Selectin FITC | P-Selectin FITC | P-Selectin FITC |
| | FITC | FITC | | | |

Experiments F, G and H

| | | | | | |
|---|---|---|---|---|---|
| IgG1 PE | HLA Class I PE | HLA Class I PE | HLA Class I PE | HLA Class I PE | HLA Class I PE |
| IgG2a FITC | HLA Class II | HLA Class II | HLA Class II | HLA Class II | HLA Class II |
| | FITC | FITC | FITC | FITC | FITC |

Experiments F and G

| | | | | | |
|---|---|---|---|---|---|
| IgG1 PE | VCAM-1 PE | VCAM-1 PE | VCAM-1 PE | VCAM-1 PE | VCAM-1 PE |

Experiment H

| | | | | | |
|---|---|---|---|---|---|
| IgG1 PE | VCAM-1 PE | VCAM-1 PE | VCAM-1 PE | VCAM-1 PE | VCAM-1 PE |
| VCAM-I PE | ICAM-2 FITC | ICAM-2 FITC | ICAM-2 FITC | ICAM-2 FITC | ICAM-2 FITC |

Experiment E

| | | | | | |
|---|---|---|---|---|---|
| IgG1 FITC | E-Selectin | E-Selectin | E-Selectin | E-Selectin | E-Selectin |
| E-Selectin FITC | P-Selectin | P-Selectin | P-Selectin | P-Selectin | P-Selectin |
| P-Selectin FITC | CD51/61 | CD5I/61 | CD51/61 | CD51/61 | CD51/61 |

Experiment I

| | | | | | |
|---|---|---|---|---|---|
| IgG2a FITC | HLA Class II | HLA Class II | HLA Class II | HLA Class II | HLA Class II |
| IgG1 PE | ICAM-1 | ICAM-1 | ICAM-1 | ICAM-1 | ICAM-1 |
| HLA Class II FITC | ICAM-2 | ICAM-2 | ICAM-2 | ICAM-2 | ICAM-2 |

Experiment J

| | | | | | |
|---|---|---|---|---|---|
| Ms IgG2a | ICAM-2 FITC | ICAM-2 FITC | ICAM-2 FITC | ICAM-2 FITC | ICAM-2 FITC |
| IgG1 FITC | CD144 | CD144 | CD144 | CD144 | CD144 |
| ICAM-2 FITC | CD44 | CD44 | CD44 | CD44 | CD44 |

Experiments K and L

| | | | | | |
|---|---|---|---|---|---|
| Ms IgG2a | CD144 | CD144 | CD144 | CD144 | CD144 |
| IgG1 FITC | CD44 | CD44 | CD44 | CD44 | CD44 |

After the antibodies were added, the tubes were incubated on ice for 30 minutes and covered with foil. Then, the tubes were centrifuged at 1200 RPM for 8 minutes at 4° C. The supernatant was carefully removed. The cells were re-suspended in 2 ml of PBS-FACS buffer and centrifuged again as above. The supernatant was carefully removed again and the cells were re-suspended in 500 μl of PBS-FACS buffer. The tubes were then analyzed using a flow cytometer. Each adhesion molecule was tested two or three times, each time using a different HUVEC donor.

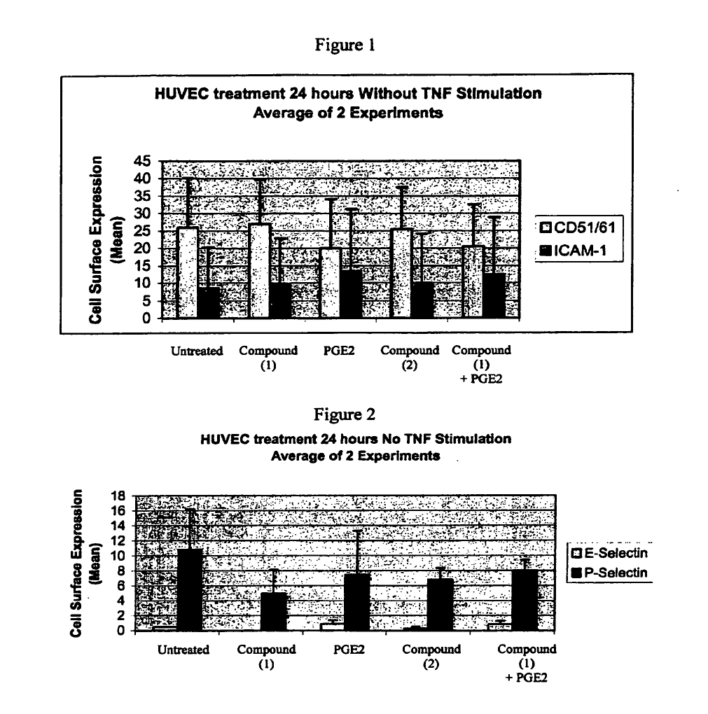

C) Results. The adhesion markers CD51/61, ICAM-1, E-Selectin and P-Selectin that are expressed on HUVEC were examined under unstimulated conditions and treated with Compound (1) (10 μM), PGE2 (10 μM), Compound (2) (10 μM) or with a mixture of Compound (1) (10 μM) and PGE2 (10 μM). In the unstimulated condition, CD51/61 cell surface expression was unaffected by either Compound (1) or Compound (2) compared to untreated conditions. Both PGE2 and the mixture of Compound (1) and PGE2 treatments resulted in a 20% reduction in the CD51/61 cell surface expression. Cell surface expression of ICAM-1 displayed a modest 10-20% increase from both Compound (1) and Compound (2) treatments. The mixture of Compound (1) and PGE2 enhanced the cell surface expression of ICAM-1 to approximately 25-30%, although the increase is less than that observed for PGE2 alone (see FIG. 1).

E-Selectin cell surface expression levels were small possibly due to insufficient sensitivity. Nevertheless, Compound (1) and Compound (2) inhibited E-Selectin expression and PGE2 seemed to block the Compound (1) induced inhibition, restoring E-Selectin expression levels to baseline values. P-Selectin expressions was also inhibited by Compound (1) and Compound (2) by approximately 55% and 35% respectively. PGE2 reduced the level of inhibition caused by Compound (1) from approximately 55% to 27% when used in combination, however the remaining expression level was similar to that of PGE2 alone (see FIG. 2).

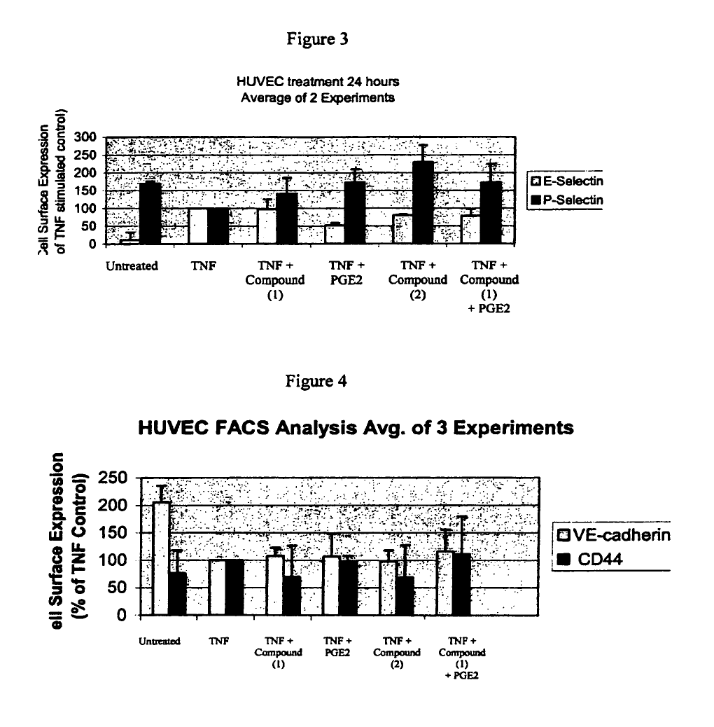

For TNF-α (1 ng/ml) stimulated conditions, cell surface expression levels of adhesion molecules were normalized as a percentage of TNF-α-stimulated expression (100%). Under this condition, the TNF-α-stimulated cells surface expression of E-Selectin was unaffected by Compound (1), whereas Compound (2) (10 μM) inhibited TNF-α-induced E-Selectin expression by approximately 20%. PGE2 alone resulted in a 50% reduction in the TNF-α-induced E-Selectin expression. The addition of Compound (1) reduced the PGE2 mediated blockade of E-Selectin (see FIG. 3). Both Compound (1) and Compound (2) increased the TNF-α-induced cell expression of P-Selectin to 40% and >2 fold above baseline respectively. The mixture of Compound (1) and PGE2 increased TNF-α-stimulated cell expression of P-Selectin to levels comparable to PGE2 alone (see FIG. 3).

The TNF-α-stimulated cell surface expression of VE-Cadherin was unaffected by Compound (1), Compound (2), PGE2 or the mixture of Compound (1) and PGE2 (see FIG. 4). However, the TNF-α-stimulated cell surface expression of CD44 was inhibited approximately 30% by both Compound (1) and Compound (2). Although PGE2 alone had no detectable effects, the mixture of Compound (1) and PGE2 eliminated the 30% inhibition observed with Compound (1) alone and restored expression level to that of PGE2 alone which were comparable to baseline levels (see FIG. 4).

Several adhesion markers expressed on HUVEC were examined in the TNF-α stimulated condition in conjunction with Compound (1) (10 μM), PGE2 (10 μM), Compound (2) (10 μM) or with the mixture of Compound (1) and PGE2. Among the panel tested, Compound (1) treatment increased ICAM-1 and P-Selectin cell surface expression by approximately 35% and decreased VCAM expression by 30%. Using the same test markers, Compound (2) increased P-Selectin expression nearly 2 fold. PGE2 and the mixture of Compound (1) and PGE2 significantly decreased the cell surface expression of both VCAM and E-Selectin. The reductions observed were comparable to PGE2 alone suggesting a mechanism that does not involve Compound (1) phosphodiesterase inhibition (see FIG. 5).

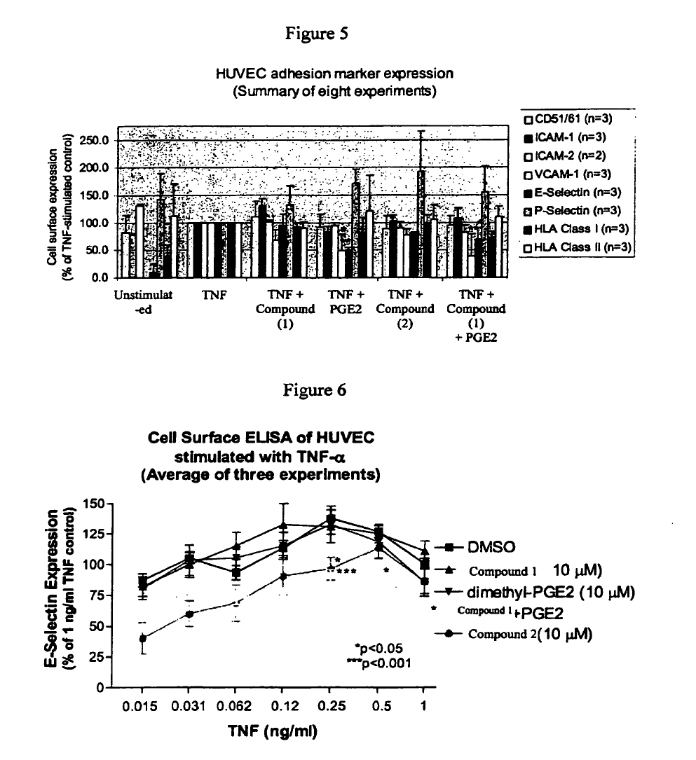

Using ELISA to detect E-Selectin cells surface expression in HUVEC following TNF-α stimulator demonstrated that the mixture of Compound (1) and PGE2 (10 μM) significantly inhibited expression at 0.25, 0.5 and 1 ng/ml of TNF-α compared to either agent alone. In this assay, the mixture of Compound (1) and PGE2 appeared to work synergistically. Also, Compound (2) displays an inhibitory effect on TNF-α-stimulated E-Selectin cell surface expression (see FIG. 6).

### Example 16

### Study for Ultraviolet B-induced TNF-alpha Production by Human Keratinocytes

Cutaneous lupus patients often experience disease exacerbation when exposed to ultraviolet (UV) light. This is thought to be due to UVB-induced TNF-α production by keratinocytes. Keratinocytes have been shown to release cytokines including TNF-α after exposure to low levels of UVB radiation in vitro (Takashima, 1996). In vitro study for cutaneous lupus was performed to investigate how the compounds of the invention affect TNF-α production in keratinocytes.

Human neonatal foreskin epidermal keratinocytes (HEKn cells) were obtained from Cascade Biologics and were grown in serum-free medium supplemented with growth factors. When cells reached 80% confluency, they were trypsinized and plated at 1×10⁵ cells/well in 6 well dishes. Plates were incubated for 24 hours to allow cell adhesion. To optimize conditions for the release of TNF-α, cells were treated with various degrees of exposure to UVB radiation (1, 4 and 24 hours). Supernatants were then collected and tested in the TNF-α ELISA.

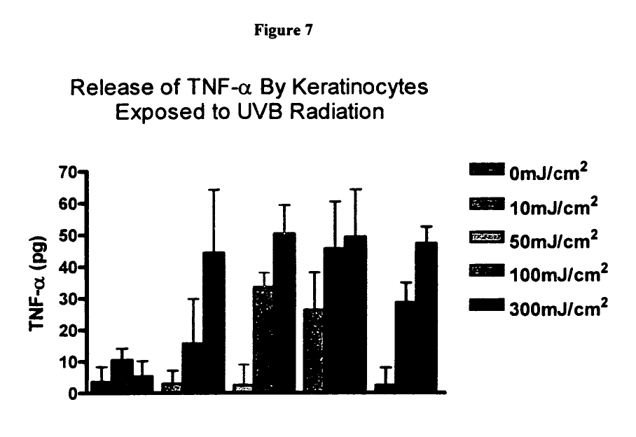

FIG. 7 shows that HEKn cells were treated with 0, 10, 50, 100, or 300 mJ/cm² UVB radiation. Supernatants were collected and tested in the TNF-α ELISA at 1 Hour (bars with no pattern), 4 Hours (lined bars), or 24 Hours (checkered bars) after exposure. Results are the average of two experiments. The results shown in FIG. 7 indicate that supernatants collected 24 hours after HEKn cells were exposed to UVB had the highest levels of TNF-α. After exposure to 10, 50, or 100 mJ/cm², cells remained attached to the wells and no cell damage was observed. The 300 mJ/cm2 UVB exposure was too high for the HEKn cells and many cells were damaged and detached from the bottom of the wells. Based on these results, future experiments concentrated on testing the effect of the compounds of the invention on TNF-α levels 24 hours after HEKn cells are exposed to 50 mJ/cm² radiation.

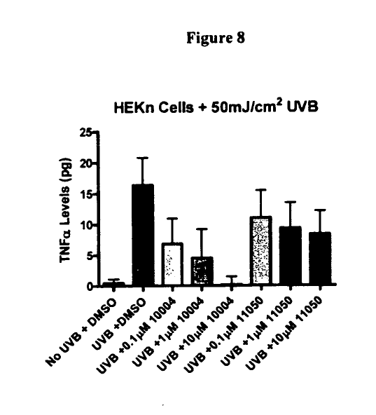

Cells were treated for 4 hours with (+)-2-[1-(3-ethoxy-4-methoxyphenyl)-2-methylsulfonylethyl]-4-acetylaminoisoindoline-1,3-dione (compound named 10004), or cyclopropyl {2-[(1S)-1-(3-ethoxy-4-methoxyphenyl)-2-(methylsulfonyl)ethyl]-3-oxoisoindolin-4-yl}carboxamide (compound named 11050) at 0.1 μM, 1 μM, or 10 μM. Medium was aspirated the cells washed and then exposed to 50 mJ/cm² UVB radiation. Fresh medium was added and the cells were incubated for 24 hours. Supernatents were removed and tested in the TNF-α ELISA kit from Pierce Biotechnology. Cells treated with the compounds showed a dose dependent decrease in levels of TNF-α released after UVB exposure. (FIG. 8). (+)-2-[1-(3-ethoxy-4-methoxyphenyl)-2-methylsulfonylethyl]-4-acetylaminoisoindoline-1,3-dione (compound named 10004) had a greater effect on TNF-α levels with the 10 μM treatment, showing TNF-α levels similar to cells not treated with radiation. (FIG. 8).

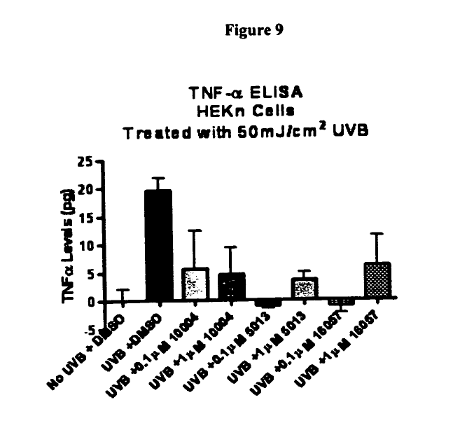

In other experiments, cells were treated for 4 hours with 3-(4-amino-1-oxo-1,3-dihydro-isoindol-2-yl)-piperidine-2,6-dione (compound named 5013), (+)-2-[1-(3-ethoxy-4-methoxyphenyl)-2-methylsulfonylethyl]-4-acetylaminoisoindoline-1,3-dione (compound named 10004) or compound named 16057, at 0.1 μM or 1 μM. Cells treated with (+)-2-[1-(3-ethoxy-4-methoxyphenyl)-2-methylsulfonylethyl]-4-acetylaminoisoindoline-1,3-dione (compound named 10004) showed a dose dependent decrease in levels of TNF-α released after UVB exposure. (FIG. 9). 3-(4-Amino-1-oxo-1,3-dihydro-isoindol-2-yl)-piperidine-2,6-dione (compound named 5013) had a greater effect on TNF-α levels with the 0.1 μM treatment showing TNF-α levels lower than cells not treated with radiation. (FIG. 9).

The embodiments of the invention described above are intended to be merely exemplary and those skilled in the art will recognize or will be able to ascertain using no more than routine experimentation, numerous equivalents of specific compounds, materials and procedures. All such equivalents are considered to be within the scope of the invention and are encompassed by the appended claims.

What is claimed is:

1. A method of treating cutaneous lupus in a human, which comprises administering to a patient having cutaneous lupus a therapeutically effective amount of (+)-2-[1-(3-ethoxy-4-methoxyphenyl)-2-methylsulfonylethyl]-4-acetylaminoisoindoline-1,3-dione, or a pharmaceutically acceptable salt or solvate thereof, substantially free of its (−) enantiomer.

2. A method of treating cutaneous lupus, which comprises administering to a patient having cutaneous lupus a therapeutically effective amount of 4-(amino)-2-(2,6-dioxo(3-piperidyl))-isoindoline-1,3-dione, or a pharmaceutically acceptable salt, solvate or stereoisomer thereof.

3. A method of treating cutaneous lupus, which comprises administering to a patient having cutaneous lupus a therapeutically effective amount of 3-(4-amino-1-oxo-1,3-dihydro-isoindol-2-yl)-piperidine-2,6-dione, or a pharmaceutically acceptable salt, solvate or stereoisomer thereof.

4. A method of treating cutaneous lupus in a human, which comprises administering to a patient having cutaneous lupus a therapeutically effective amount of cyclopropyl {2-[(1S)-1-(3-ethoxy-4-methoxyphenyl)-2-(methylsulfonyl)ethyl]-3-oxoisoindolin-4-yl}carboxamide, or a pharmaceutically acceptable salt or solvate thereof, substantially free of its (R) enantiomer.

5. The method of claim 1, 2, 3 or 4, wherein the compound is administered as a pharmaceutically acceptable salt.

6. The method of claim 1, 2, 3 or 4, wherein the compound is administered as a pharmaceutically acceptable solvate.

7. The method of claim 6, wherein the solvate is a hydrate.

8. The method of claim 2 or 3, wherein the compound is R-enantiomer.

9. The method of claim 2 or 3, wherein the compound is S-enantiomer.

10. The method of claim 1, 2, 3 or 4, wherein the cutaneous lupus is acute cutaneous lupus erythematosus.

11. The method of claim 1, 2, 3 or 4, wherein the cutaneous lupus is subacute cutaneous lupus erythematosus.

12. The method of claim 1, 2, 3 or 4, wherein the cutaneous lupus is discoid lupus erythematosus.

13. The method of claim 1, 2, 3 or 4, wherein the cutaneous lupus is neonatal lupus erythematosus.

14. The method of claim 1, 2, 3 or 4, wherein the cutaneous lupus is lupus erythematosus of childhood.

15. The method of claim 1, 2, 3 or 4, further comprising administering to the patient a therapeutically effective amount of a second active agent.

16. The method of claim 15, wherein the second active agent is an anti-inflammatory, an immunomodulatory compound, an anti-malarial, an immunosuppressant, an antibiotic, an antiviral, an immunoglobulin, an immunologic-enhancing drug, a hormone, PGE2 or a combination thereof.

17. The method of claim 16, wherein the second active agent is PGE2.

18. The method of claim 1, 2, 3 or 4, wherein the compound or a pharmaceutically acceptable salt, solvate or stereoisomer thereof is administered orally.

19. The method of claim 1, 2, 3 or 4, wherein the compound or a pharmaceutically acceptable salt, solvate or stereoisomer thereof is administered parenterally.

20. The method of claim 1, 2, 3 or 4, wherein the compound or a pharmaceutically acceptable salt, solvate or stereoisomer thereof is administered topically.

21. The method of claim 20, wherein the compound or a pharmaceutically acceptable salt, solvate or stereoisomer thereof is administered topically in a dosage form of ointment, cream, gel, paste, dusting powder, lotion, spray, liniment, poultice, aerosol, solution, emulsion or suspension.

22. The method of claim 1, 2, 3 or 4, wherein the compound or a pharmaceutically acceptable salt, solvate or stereoisomer thereof is administered orally in a dosage form of a tablet or a capsule.

23. The method of claim 22, wherein the compound or a pharmaceutically acceptable salt, solvate or stereoisomer thereof is administered orally in 5 mg, 10 mg, 15 mg or 25 mg of a tablet or a capsule.

24. The method of claim 1 or 4, wherein the therapeutically effective amount is from about 1 mg to about 1000 mg per day.

25. The method of claim 24, wherein the therapeutically effective amount is from about 5 mg to about 500 mg per day.

26. The method of claim 25, wherein the therapeutically effective amount is from about 10 mg to about 200 mg per day.

27. The method of claim 2 or 3, wherein the therapeutically effective amount is from about 0.1 to about 150 mg per day.

28. The method of claim 27, wherein the therapeutically effective amount is from about 1 to about 100 mg per day.

29. The method of claim 27, wherein the therapeutically effective amount is about 0.1, 1, 2, 5, 10, or 25 mg per day.

30. The method of claim 2, wherein the therapeutically effective amount is from about 0.5 to about 2 mg per day.

31. The method of claim 2, wherein the therapeutically effective amount is about 5 mg every other day.

32. The method of claim 3, wherein the therapeutically effective amount is from about 5 to about 25 mg per day.

33. The method of claim 3, wherein the therapeutically effective amount is about 25 mg or 50 mg every other day.

34. The method of claim 1, 2, 3 or 4, wherein the compound or a pharmaceutically acceptable salt, solvate or stereoisomer thereof is administered once or twice per day.

35. The method of claim 1, 2, 3 or 4, wherein the compound or a pharmaceutically acceptable salt, solvate or stereoisomer thereof is administered cyclically.
